(19) United States
(12) Patent Application Publication
(10) Pub. No.: US 2026/0166175 A1
(43) Pub. Date: Jun. 18, 2026

(54) FABRICATION OF CYCLODEXTRIN PARTICLES AND CRYOGELS AND METHODS OF USING AND MAKING THEREOF

(71) Applicant: University of South Florida, Tampa, FL (US)

(72) Inventors: Nurettin SAHINER, Wesley Chapel, FL (US); Manoj Kumar RAM, Palm Harbor, FL (US); Ramesh S. AYYALA, Tampa, FL (US)

(21) Appl. No.: 19/124,409

(22) PCT Filed: Oct. 27, 2023
(86) PCT No.: PCT/US2023/078046

§ 371 Date: Apr. 25, 2025

Related U.S. Application Data
(60) Provisional application No. 63/381,189, filed on Oct. 27, 2022.

(51) Int. Cl. A61K 47/69; A61K 45/06

(52) U.S. Cl. CPC A61K 47/6951; A61K 45/06; A61K 2300/00

(57) ABSTRACT

The present disclosure provides for a polymer comprising repeating units of a cyclodextrin comprising α-cyclodextrin (α-CD), γ-cyclodextrin (γ-CD), or any combination thereof, and a crosslinker. Further provided herein are a particle and cryogel comprising the same. Also provided herein are methods of using and making thereof.

### CROSS-REFERENCE TO RELATED APPLICATIONS

This application claims the benefit of priority to U.S. Provisional Application No. 63/381,189 filed on Oct. 27, 2022, the disclosure of which is hereby expressly incorporated by reference herein in its entirety.

### BACKGROUND

Certain drugs used for treatment of varying diseases and disorders have low solubility, bioavailability, and physical and chemical stability in biological environments. This can result in challenges when treating diseases and disorders in a subject. Furthermore, these drugs can be more effective when administered slowly over time. There is also a need for a means for encapsulating other materials such as dyes and toxic compounds so as to remove them and transport them to minimize harm to plants, animals, and humans.

The compositions and methods disclosed herein address these and other needs.

### SUMMARY

In accordance with the purposes of the disclosed materials and methods, as embodied and broadly described herein, the disclosed subject matter, in one aspect, relates to cyclodextrins and uses and the making thereof.

Thus, in one example, a polymer is provided, including repeating units of a cyclodextrins comprising α-cyclodextrin (α-CD), γ-cyclodextrin (γ-CD), or any combination thereof, and a crosslinker.

In a further example, a particle is provided, including the polymer disclosed herein.

Additionally, a cryogel is provided, including the polymer disclosed herein.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying figures, which are incorporated in and constitute a part of this specification, illustrate several aspects described below.

FIGS. 1A-1B show (FIG. 1A) the reaction schema of CD particle preparation from α-, β-, or γ-Cyclodextrin (FIG. 1A) and SEM images of poly(α-Cyclodextrin) (p(α-CD), poly(β-Cyclodextrin) (p(β-CD)), or pol(γ-Cyclodextrin) (p(γ-CD)) particles (FIG. 1B).

FIG. 2 shows FT-IR spectra of α-CD, p(α-CD) particles, β-CD, p(β-CD) particles, and γ-CD, and p(γ-CD) particles.

FIGS. 3A-3C show thermogravimetric analysis of α-CD and p(α-CD) particles (FIG. 3A), R-CD and p(β-CD) particles (FIG. 3B), and γ-CD and p(γ-CD) particles (FIG. 3C).

FIGS. 4A-4C show cytotoxicity of α-CD molecules and p(α-CD) particles (FIG. 4A), β-CD molecules and p(β-CD) particles (FIG. 4B), and γ-CD molecules and p(γ-CD) particles (FIG. 4C) on L929 fibroblasts at 24 h incubation time.

FIG. 5A-5B show hemolysis (FIG. 5A) and blood clotting (FIG. 5B) index values of 1 mg/mL concentration of α-CD, β-CD, γ-CD, and p(α-CD) particles, p(β-CD) particles, and p(γ-CD) particles.

FIGS. 6A-6B show the calibration curves of 10-50 mg/L, 3 mL of BPA solution in 1:7 volume ratio of ethanol:DI water mixture at 275 nm wavelength (FIG. 6A), and 0.625-10 mg/L, 3 mL of CUR solution in 3:2 volume ratio of ethanol:DI water mixture at 425 nm wavelength (FIG. 6B).

FIG. 7 shows the change in zeta potential (ζ, mV) values with solution pH of p(α-CD), p(β-CD), and p(γ-CD) particles.

FIGS. 8A-8B show survival yield curves for (FIG. 8A) BPA; [α-CD+BPA-H]⁻, [(3-CD+BPA-H]⁻ and [γ-CD+BPA-H]⁻ with Center-of-Mass Energies (E_{CM}) about 0.274 eV for [α-CD+BPA-H]⁻, 0.272 eV [(β-CD+BPA-H]⁻ and 0.250 eV [γ-CD+BPA-H]⁻. (FIG. 8B) CUR; [α-CD+CUR-H]⁻, [β-CD+CUR-H]⁻ and [γ-CD+CUR-H]⁻ with ECM50 about 0.505 eV for [α-CD+CUR-H]⁻, 0.562 eV [(β-CD+CUR-H]⁻ and 0.508 eV [γ-CUR+CUR-H]⁻.

FIGS. 9A-9D show (FIG. 9A) Bisphenol A (BPA) and (FIG. 9B) Curcumin (CUR) release profiles of p(α-CD), p(β-CD), and p(γ-CD) particles in ethanol solution. (FIG. 9C) Bisphenol A and (FIG. 9D) Curcumin release profiles of p(α-CD), p(β-CD), and p(γ-CD) particles in PBS.

FIG. 10 shows schematic representation of the complex formation of (top) BPA and (bottom) CUR with CD rings.

FIGS. 11A-11C show Ciprofloxacin (FIG. 11A), methyl orange (FIG. 11B) or methylene blue (FIG. 11C) release from p(α-CD), p(β-CD) and p(γ-CD) particles at 37° C. and physiological pH condition (pH 7.4).

FIG. 12 shows chemical structures of cyclodextrins and copolymeric p(CD) particles.

FIG. 13 shows SEM images of p(β-CD)-1, -2, and -3 cryogels.

### DETAILED DESCRIPTION

The following description of the disclosure is provided as an enabling teaching of the disclosure in its best, currently known embodiments. Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As can be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.”

As used in the specification and the appended claims, the singular forms “a,” “an” and “the” include plural referents unless the context clearly dictates otherwise. Thus, for example, reference to “a compound”, “a composition”, or “a disorder”, includes, but is not limited to, two or more such compounds, compositions, or disorders, and the like.

It should be noted that ratios, concentrations, amounts, and other numerical data can be expressed herein in a range format. It can be further understood that the endpoints of each of the ranges are significant both in relation to the other endpoint, and independently of the other endpoint.

The term “characteristic dimension,” as used herein, refers to the largest straight-line distance between two points in the plane of a cross-sectional shape. Herein, the plane of a cross-sectional shape can be that of a liner or cap, for example. “Average characteristic dimension” generally refers to the statistical mean characteristic dimension. For example, when the liner or cap has a cross-sectional shape that is substantially circular, the average characteristic dimension can refer to the average diameter.

The term “subject” preferably refers to a human in need of treatment with an anti-cancer agent or treatment for any purpose, and more preferably a human in need of such a treatment to treat cancer, or a precancerous condition or lesion. However, the term “patient” can also refer to non-human animals, preferably mammals such as dogs, cats, horses, cows, pigs, sheep, and non-human primates, among others, that need treatment with an anti-cancer agent or treatment.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

### Chemical Definitions

Unless otherwise defined, all technical and scientific terms used herein have the same meaning as commonly understood by one of ordinary skill in the art to which this invention belongs.

The organic moieties mentioned when defining variable positions within the general formulae described herein (e.g., the term “halogen”) are collective terms for the individual substituents encompassed by the organic moiety. The prefix Cₙ-Cₘ preceding a group or moiety indicates, in each case, the possible number of carbon atoms in the group or moiety that follows.

The term “ion,” as used herein, refers to any molecule, portion of a molecule, cluster of molecules, molecular complex, moiety, or atom that contains a charge (positive, negative, or both at the same time within one molecule, cluster of molecules, molecular complex, or moiety (e.g., zwitterions)) or that can be made to contain a charge. Methods for producing a charge in a molecule, portion of a molecule, cluster of molecules, molecular complex, moiety, or atom are disclosed herein and can be accomplished by methods known in the art, e.g., protonation, deprotonation, oxidation, reduction, alkylation, acetylation, esterification, de-esterification, hydrolysis, etc.

The term “anion” is a type of ion and is included within the meaning of the term “ion.” An “anion” is any molecule, portion of a molecule (e.g., zwitterion), cluster of molecules, molecular complex, moiety, or atom that contains a net negative charge or that can be made to contain a net negative charge. The term “anion precursor” is used herein to specifically refer to a molecule that can be converted to an anion via a chemical reaction (e.g., deprotonation).

The term “cation” is a type of ion and is included within the meaning of the term “ion.” A “cation” is any molecule, portion of a molecule (e.g., zwitterion), cluster of molecules, molecular complex, moiety, or atom, that contains a net positive charge or that can be made to contain a net positive charge. The term “cation precursor” is used herein to specifically refer to a molecule that can be converted to a cation via a chemical reaction (e.g., protonation or alkylation).

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

“Z¹,” “Z²,” “Z³,” and “Z⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” as used herein refers to a non-aromatic hydrocarbon group and includes branched and unbranched, alkyl, alkenyl, or alkynyl groups.

As used herein, the term “alkyl” refers to saturated, straight chained or branched saturated hydrocarbon moieties. Unless otherwise specified, C₁-C₂₄ (e.g., C₁-C₂₂, C₁-C₂₀, C₁-Cis, C₁-C₁₆, C₁-C₁₄, C₁-C₁₂, C₁-C₁₀, C₁-C₅, C₁-C₆, or C₁-C₄) alkyl groups are intended. Examples of alkyl groups include methyl, ethyl, propyl, 1-methyl-ethyl, butyl, 1-methyl-propyl, 2-methyl-propyl, 1,1-dimethyl-ethyl, pentyl, 1-methyl-butyl, 2-methyl-butyl, 3-methyl-butyl, 2,2-dimethyl-propyl, 1-ethyl-propyl, hexyl, 1,1-dimethyl-propyl, 1,2-dimethyl-propyl, 1-methyl-pentyl, 2-methyl-pentyl, 3-methyl-pentyl, 4-methyl-pentyl, 1,1-dimethyl-butyl, 1,2-dimethyl-butyl, 1,3-dimethyl-butyl, 2,2-dimethyl-butyl, 2,3-dimethyl-butyl, 3,3-dimethyl-butyl, 1-ethyl-butyl, 2-ethyl-butyl, 1,1,2-trimethyl-propyl, 1,2,2-trimethyl-propyl, 1-ethyl-1-methyl-propyl, 1-ethyl-2-methyl-propyl, heptyl, octyl, nonyl, decyl, dodecyl, tetradecyl, hexadecyl, eicosyl, tetracosyl, and the like. Alkyl substituents may be unsubstituted or substituted with one or more chemical moieties. The alkyl group can be substituted with one or more groups including, but not limited to, hydroxyl, halogen, acetal, acyl, alkyl, alkoxy, alkenyl, alkynyl, aryl, heteroaryl, aldehyde, amino, cyano, carboxylic acid, ester, ether, carbonate ester, carbamate ester, ketone, nitro, phosphonyl, silyl, sulfo-oxo, sulfonyl, sulfone, sulfoxide, or thiol, as described below, provided that the substituents are sterically compatible and the rules of chemical bonding and strain energy are satisfied.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halides (halogens; e.g., fluorine, chlorine, bromine, or iodine). The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

As used herein, the term “alkenyl” refers to unsaturated, straight chained, or branched hydrocarbon moieties containing a double bond. Unless otherwise specified, C₂-C₂₄ (e.g., C₂-C₂₂, C₂-C₂₀, C₂-C₁₈, C₂-C₁₆, C₂-C₁₄, C₂-C₁₂, C₂-C₁₀, C₂-C₅, C₂-C₆, or C₂-C₄) alkenyl groups are intended. Alkenyl groups may contain more than one unsaturated bond. Examples include ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, and 1-ethyl-2-methyl-2-propenyl. The term “vinyl” refers to a group having the structure —CH═CH₂; 1-propenyl refers to a group with the structure —CH═CH—CH₃; and 2-propenyl refers to a group with the structure —CH₂—CH═CH₂. Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C. Alkenyl substituents may be unsubstituted or substituted with one or more chemical moieties. Examples of suitable substituents include, for example, alkyl, alkoxy, alkenyl, alkynyl, aryl, heteroaryl, acetal, acyl, aldehyde, amino, cyano, carboxylic acid, ester, ether, carbonate ester, carbamate ester, halide, hydroxyl, ketone, nitro, phosphonyl, silyl, sulfo-oxo, sulfonyl, sulfone, sulfoxide, or thiol, as described below, provided that the substituents are sterically compatible and the rules of chemical bonding and strain energy are satisfied.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, acetal, acyl, aldehyde, amino, cyano, carboxylic acid, ester, ether, carbonate ester, carbamate ester, halide, hydroxyl, ketone, nitro, phosphonyl, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems (e.g., monocyclic, bicyclic, tricyclic, polycyclic, etc.) that can be substituted or unsubstituted. A cyclic group can contain one or more aryl groups, one or more non-aryl groups, or one or more aryl groups and one or more non-aryl groups.

The term “acyl” as used herein is represented by the formula —C(O)Z‘ where Z’ can be a hydrogen, hydroxyl, alkoxy, alkyl, alkenyl, alkynyl, aryl, heteroaryl, cycloalkyl, cycloalkenyl, heterocycloalkyl, or heterocycloalkenyl group described above. As used herein, 5 the term “acyl” can be used interchangeably with “carbonyl.” Throughout this specification “C(O)” or “CO” is a shorthand notation for C═O.

The term “alkanol” as used herein is represented by the formula Z¹OH, where Z¹ can be an alkyl, alkenyl, alkynyl, aryl, heteroaryl, cycloalkyl, cycloalkenyl, heterocycloalkyl, or heterocycloalkenyl group described above.

As used herein, the term “alkoxy” as used herein is an alkyl group bound through a single, terminal ether linkage; that is, an “alkoxy” group can be defined as to a group of the formula Z¹—O—, where Z¹ is unsubstituted or substituted alkyl as defined above. Unless otherwise specified, alkoxy groups wherein Z¹ is a C₁-C₂₄ (e.g., C₁-C₂₂, C₁-C₂₀, C₁-C₁₈, C₁-C₁₆, C₁-C₁₄, C1-C₁₂, C₁-C₁₀, C₁-C₅, C₁-C₆, or C1-C₄) alkyl group are intended. Examples include methoxy, ethoxy, propoxy, 1-methyl-ethoxy, butoxy, 1-methyl-propoxy, 2-methyl-propoxy, 1,1-dimethyl-ethoxy, pentoxy, 1-methyl-butyloxy, 2-methyl-butoxy, 3-methyl-butoxy, 2,2-di-methyl-propoxy, 1-ethyl-propoxy, hexoxy, 1,1-dimethyl-propoxy, 1,2-dimethyl-propoxy, 1-methyl-pentoxy, 2-methyl-pentoxy, 3-methyl-pentoxy, 4-methyl-penoxy, 1,1-dimethyl-butoxy, 1,2-dimethyl-butoxy, 1,3-dimethyl-butoxy, 2,2-dimethyl-butoxy, 2,3-dimethyl-butoxy, 3,3-dimethyl-butoxy, 1-ethyl-butoxy, 2-ethylbutoxy, 1,1,2-trimethyl-propoxy, 1,2,2-trimethyl-propoxy, 1-ethyl-1-methyl-propoxy, and 1-ethyl-2-methyl-propoxy.

The term “ether” as used herein is represented by the formula Z¹OZ², where Z¹ and Z² can be, independently, an alkyl, alkenyl, alkynyl, aryl, heteroaryl, cycloalkyl, cycloalkenyl, heterocycloalkyl, or heterocycloalkenyl group described above.

The term “epoxy” or “epoxide” as used herein refers to a cyclic ether with a three-atom ring and can represented by the formula:

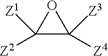

where Z¹, Z², Z³, and Z⁴ can be, independently, an alkyl, alkenyl, alkynyl, aryl, heteroaryl, cycloalkyl, cycloalkenyl, heterocycloalkyl, or heterocycloalkenyl group described above.

The term “hydroxyl” as used herein is represented by the formula —OH.

The term “silyl” as used herein is represented by the formula —SiZ¹Z²Z³, where Z¹, Z², and Z³ can be, independently, hydrogen, alkyl, alkoxy, alkenyl, alkynyl, aryl, heteroaryl, cycloalkyl, cycloalkenyl, heterocycloalkyl, or heterocycloalkenyl group described above.

The term “sulfonyl” or “sulfone” is used herein to refer to the sulfo-oxo group represented by the formula —S(O)₂Z¹, where Z¹ can be hydrogen, an alkyl, alkenyl, alkynyl, aryl, heteroaryl, cycloalkyl, cycloalkenyl, heterocycloalkyl, or heterocycloalkenyl group described above.

The term “sulfide” as used herein comprises the formula —S—.

“R¹,” “R²,” “R3,” “Rⁿ,” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an amine group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

Unless stated to the contrary, a formula with chemical bonds shown only as solid lines and not as wedges or dashed lines contemplates each possible stereoisomer or mixture of stereoisomer (e.g., each enantiomer, each diastereomer, each meso compound, a racemic mixture, or scalemic mixture).

### Composition

### Polymer

Provided herein is a polymer comprising repeating units of a cyclodextrin comprising α-cyclodextrin (α-CD), γ-cyclodextrin (γ-CD), or any combination thereof, and a crosslinker.

In some examples, the cyclodextrin further comprises β-cyclodextrin (β-CD).

Cyclodextrins are a family of cyclic oligosaccharides with a macrocyclic ring of glucose subunits joined by α-1,4 glycosidic bonds.

In some examples, cyclodextrins include α-cyclodextrin (α-CD; alpha-CD), β-cyclodextrin (β-CD; beta-CD), γ-cyclodextrins (γ-CD; gamma-CD), or any combination thereof.

α-CD is an oligosaccharide derived from glucose and contains six glucose units. The six glucose subunits are linked end to end via α-1,4 linkages. This results in the cyclodextrin having the shape of a tapered cylinder, with six primary alcohols on one face and twelve secondary alcohol groups on the other. Alpha-CD has the following chemical structure:

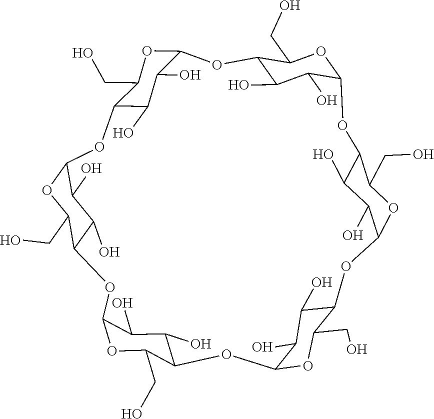 [SMILES: OCC1OC2OC3C(CO)OC(OC4C(CO)OC(OC5C(CO)OC(OC6C(CO)OC(OC7C(CO)OC(OC1C(O)C2O)C(O)C7O)C(O)C6O)C(O)C5O)C(O)C4O)C(O)C3O]

β-CD is an oligosaccharide derived from glucose and contains seven glucose units. The seven glucose subunits are linked end to end via α-1,4 linkages. This results in the cyclodextrin having the shape of a tapered cylinder, with seven primary alcohols on one face and fourteen secondary alcohol groups on the other. In some examples, beta-CD is used in addition to alpha-CD and/or gamma-CD to create a copolymer or terpolymer. Beta-CD has the following chemical structure:

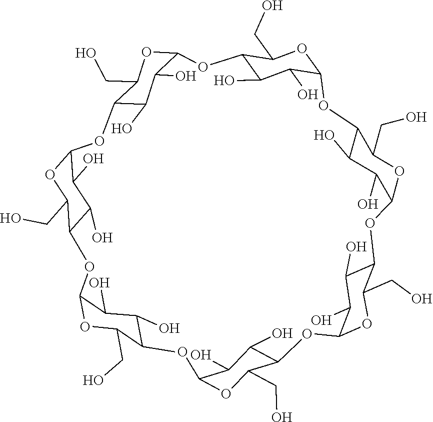 [SMILES: OCC1OC2OC3C(CO)OC(OC4C(CO)OC(OC5C(CO)OC(OC6C(CO)OC(OC7C(CO)OC(OC8C(CO)OC(OC1C(O)C2O)C(O)C8O)C(O)C7O)C(O)C6O)C(O)C5O)C(O)C4O)C(O)C3O]

γ-CD is an oligosaccharide derived from glucose and contains eight glucose units. The eight glucose subunits are linked end to end via α-1,4 linkages. This results in the cyclodextrin having the shape of a tapered cylinder, with eight primary alcohols on one face and sixteen secondary alcohol groups on the other. Gamma-CD has the following chemical structure:

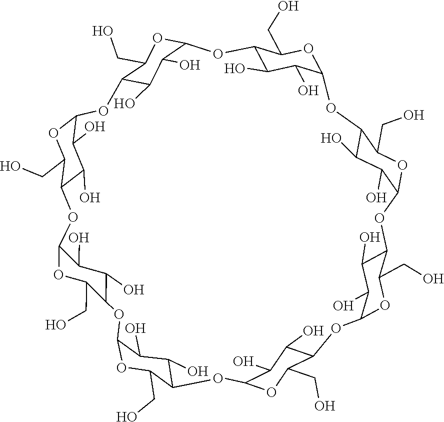 [SMILES: OCC1OC2OC3C(CO)OC(OC4C(CO)OC(OC5C(CO)OC(OC6C(CO)OC(OC7C(CO)OC(OC8C(CO)OC(OC9C(CO)OC(OC1C(O)C2O)C(O)C9O)C(O)C8O)C(O)C7O)C(O)C6O)C(O)C5O)C(O)C4O)C(O)C3O]

The polymer disclosed herein has the following formula:

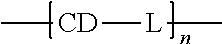

wherein CD represents α-GD or γ-CD;
wherein L represents a divalent linker; and
where n is 2 to 100,000.

In some examples, CD represents β-CD.

Crosslinkers (or crosslinking reagents) are molecules containing two or more reactive ends capable of chemically attaching to specific functional groups on other molecules. Crosslinkers are used for the process of crosslinking in which two or more molecules are chemically joined by a covalent bond.

In some examples, the polymer is a reaction product of the cyclodextrin and the crosslinker, wherein the crosslinker comprises an epoxide, a polyacrylic ester, a polycarbonate acid, a polyamidoamine, a tetrafluoroterephthalatenitrile (TFTN), divinyl sulfone (DVS), trimethylolpropanetriglycidyl ether (TMPGDE), or any combination thereof.

In further examples, the crosslinker comprises DVS. DVS, C₄H₆O₂S, is a clear colorless liquid having two S-vinyl substituents. DVS has the following chemical structure:

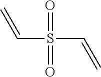

In certain examples, the crosslinker comprises TMPGDE. TMPGDE, C₁₅H₂₆O₆, is an organic chemical in the glycidyl ether family. TMPGDE has the following chemical structure:

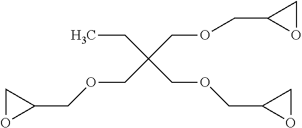 [SMILES: CCC(COCC1CO1)(COCC1CO1)COCC1CO1]

Epoxide crosslinkers are crosslinkers that include an epoxide group at the reactive ends. A general chemical structure for epoxide crosslinkers is provided below.

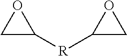

In some examples, the cyclodextrin comprises α-CD and the crosslinker comprises DVS. In further examples, the cyclodextrin comprises γ-CD and the crosslinker comprises DVS. In certain examples, the cyclodextrin comprises α-CD and the crosslinker comprises TMPGDE. In specific examples, the cyclodextrin comprises γ-CD and the crosslinker comprises TMPGDE.

In some examples, the cyclodextrin comprises α-CD and γ-CD and the cross-linker comprises DVS. The polymer of any one of claims 1-5, wherein the cyclodextrin comprises α-CD and β-CD and the cross-linker comprises DVS. In further examples, the cyclodextrin comprises β-CD and γ-CD and the cross-linker comprises DVS. In certain examples, the cyclodextrin comprises α-CD and γ-CD and the cross-linker comprises TMPGDE. In specific examples, the cyclodextrin comprises α-CD and β-CD and the cross-linker comprises TMPGDE. In some examples, the cyclodextrin comprises β-CD and γ-CD and the cross-linker comprises TMPGDE.

In further examples, the cyclodextrin comprises α-CD, β-CD, and γ-CD and the cross-linker comprises DVS. In certain examples, the cyclodextrin comprises α-CD, β-CD, and γ-CD and the cross-linker comprises TMPGDE.

### Particle

Also provided herein is a particle comprising the polymer disclosed herein.

In some examples, the particle further comprises a therapeutically effective amount of a drug. In further examples, the drug is hydrophobic. Hydrophobic is used herein to refer to the property of water repellency, opposed to water absorbency. Hydrophobic drugs have limited solubility which can create issues in administering drugs and treating health issues in a subject. Hydrophobic drugs present a solubility problem in biological environments due to enzymes, acids, etc., but administering drugs using the particles disclosed herein overcomes this problem and allows for effective administration of the drug.

In certain examples, the hydrophobic drug comprises ciprofloxacin, nitrofurantoin, propranolol, a camphotosin analog, silbylin, docetaxel, doxorubicin, naproxen, trimethoprim/sulfamethoxazole, or any combination thereof.

In some examples, lipophilic and/or hydrophilic drugs are loaded into the CD particles/cryogels. Using different crosslinkers allows for lipophilic and/or hydrophilic drug loading. In further examples, the outer surface of CD or periphery of CD units are also hydrophilic.

In further examples, the drug, or pharmaceutical agent, includes Acedapsone; Acetosulfone Sodium; Alamecin; Alexidine; Amdinocillin; Amdinocillin Pivoxil; Amicycline; Amifloxacin; Amifloxacin Mesylate; Amikacin; Amikacin Sulfate; Aminosalicylic acid; Aminosalicylate sodium; Amoxicillin; Amphomycin; Ampicillin; Ampicillin Sodium; Apalcillin Sodium; Apramycin; Aspartocin; Astromicin Sulfate; Avilamycin; Avoparcin; Azithromycin; Azlocillin; Azlocillin Sodium; Bacampicillin Hydrochloride; Bacitracin; Bacitracin Methylene Disalicylate; Bacitracin Zinc; Bambermycins; Benzoylpas Calcium; Berythromycin; Betamicin Sulfate; Biapenem; Biniramycin; Biphenamine Hydrochloride; Bispyrithione Magsulfex; Butikacin; Butirosin Sulfate; Capreomycin Sulfate; Carbadox; Carbenicillin Disodium; Carbenicillin Indanyl Sodium; Carbenicillin Phenyl Sodium; Carbenicillin Potassium; Carumonam Sodium; Cefaclor; Cefadroxil; Cefamandole; Cefamandole Nafate; Cefamandole Sodium; Cefaparole; Cefatrizine; Cefazaflur Sodium; Cefazolin; Cefazolin Sodium; Cefbuperazone; Cefdinir; Cefepime; Cefepime Hydrochloride; Cefetecol; Cefixime; Cefmenoxime Hydrochloride; Cefmetazole; Cefmetazole Sodium; Cefonicid Monosodium; Cefonicid Sodium; Cefoperazone Sodium; Ceforanide; Cefotaxime Sodium; Cefotetan; Cefotetan Disodium; Cefotiam Hydrochloride; Cefoxitin; Cefoxitin Sodium; Cefpimizole; Cefpimizole Sodium; Cefpiramide; Cefpiramide Sodium; Cefpirome Sulfate; Cefpodoxime Proxetil; Cefprozil; Cefroxadine; Cefsulodin Sodium; Ceftazidime; Ceftibuten; Ceftizoxime Sodium; Ceftriaxone Sodium; Cefuroxime; Cefuroxime Axetil; Cefuroxime Pivoxetil; Cefuroxime Sodium; Cephacetrile Sodium; Cephalexin; Cephalexin Hydrochloride; Cephaloglycin; Cephaloridine; Cephalothin Sodium; Cephapirin Sodium; Cephradine; Cetocycline Hydrochloride; Cetophenicol; Chloramphenicol; Chloramphenicol Palmitate; Chloramphenicol Pantothenate Complex; Chloramphenicol Sodium Succinate; Chlorhexidine Phosphanilate; Chloroxylenol; Chlortetracycline Bisulfate; Chlortetracycline Hydrochloride; Cinoxacin; Ciprofloxacin; Ciprofloxacin Hydrochloride; Cirolemycin; Clarithromycin; Clinafloxacin Hydrochloride; Clindamycin; Clindamycin Hydrochloride; Clindamycin Palmitate Hydrochloride; Clindamycin Phosphate; Clofazimine; Cloxacillin Benzathine; Cloxacillin Sodium; Cloxyquin; Colistimethate Sodium; Colistin Sulfate; Coumermycin; Coumermycin Sodium; Cyclacillin; Cycloserine; Dalfopristin; Dapsone; Daptomycin; Demeclocycline; Demeclocycline Hydrochloride; Demecycline; Denofungin; Diaveridine; Dicloxacillin; Dicloxacillin Sodium; Dihydrostreptomycin Sulfate; Dipyrithione; Dirithromycin; Doxycycline; Doxycycline Calcium; Doxycycline Fosfatex; Doxycycline Hyclate; Droxacin Sodium; Enoxacin; Epicillin; Epitetracycline Hydrochloride; Erythromycin; Erythromycin Acistrate; Erythromycin Estolate; Erythromycin Ethylsuccinate; Erythromycin Gluceptate; Erythromycin Lactobionate; Erythromycin Propionate; Erythromycin Stearate; Ethambutol Hydrochloride; Ethionamide; Fleroxacin; Floxacillin; Fludalanine; Flumequine; Fosfomycin; Fosfomycin Tromethamine; Fumoxicillin; Furazolium Chloride; Furazolium Tartrate; Fusidate Sodium; Fusidic Acid; Gentamicin Sulfate; Gloximonam; Gramicidin; Haloprogin; Hetacillin; Hetacillin Potassium; Hexedine; Ibafloxacin; Imipenem; Isoconazole; Isepamicin; Isoniazid; Josamycin; Kanamycin Sulfate; Kitasamycin; Levofuraltadone; Levopropylcillin Potassium; Lexithromycin; Lincomycin; Lincomycin Hydrochloride; Lomefloxacin; Lomefloxacin Hydrochloride; Lomefloxacin Mesylate; Loracarbef; Mafenide; Meclocycline; Meclocycline Sulfosalicylate; Megalomicin Potassium Phosphate; Mequidox; Meropenem; Methacycline; Methacycline Hydrochloride; Methenamine; Methenamine Hippurate; Methenamine Mandelate; Methicillin Sodium; Metioprim; Metronidazole Hydrochloride; Metronidazole Phosphate; Mezlocillin; Mezlocillin Sodium; Minocycline; Minocycline Hydrochloride; Mirincamycin Hydrochloride; Monensin; Monensin Sodiumr; Nafcillin Sodium; Nalidixate Sodium; Nalidixic Acid; Natainycin; Nebramycin; Neomycin Palmitate; Neomycin Sulfate; Neomycin Undecylenate; Netilmicin Sulfate; Neutramycin; Nifuiradene; Nifuraldezone; Nifuratel; Nifuratrone; Nifurdazil; Nifurimide; Nifiupirinol; Nifurquinazol; Nifurthiazole; Nitrocycline; Nitrofurantoin; Nitromide; Norfloxacin; Novobiocin Sodium; Ofloxacin; Onnetoprim; Oxacillin Sodium; Oximonam; Oximonam Sodium; Oxolinic Acid; Oxytetracycline; Oxytetracycline Calcium; Oxytetracycline Hydrochloride; Paldimycin; Parachlorophenol; Paulomycin; Pefloxacin; Pefloxacin Mesylate; Penamecillin; Penicillin G Benzathine; Penicillin G Potassium; Penicillin G Procaine; Penicillin G Sodium; Penicillin V; Penicillin V Benzathine; Penicillin V Hydrabamine; Penicillin V Potassium; Pentizidone Sodium; Phenyl Aminosalicylate; Piperacillin Sodium; Pirbenicillin Sodium; Piridicillin Sodium; Pirlimycin Hydrochloride; Pivampicillin Hydrochloride; Pivampicillin Pamoate; Pivampicillin Probenate; Polymyxin B Sulfate; Porfiromycin; Propikacin; Pyrazinamide; Pyrithione Zinc; Quindecamine Acetate; Quinupristin; Racephenicol; Ramoplanin; Ranimycin; Relomycin; Repromicin; Rifabutin; Rifametane; Rifamexil; Rifamide; Rifampin; Rifapentine; Rifaximin; Rolitetracycline; Rolitetracycline Nitrate; Rosaramicin; Rosaramicin Butyrate; Rosaramicin Propionate; Rosaramicin Sodium Phosphate; Rosaramicin Stearate; Rosoxacin; Roxarsone; Roxithromycin; Sancycline; Sanfetrinem Sodium; Sarmoxicillin; Sarpicillin; Scopafungin; Sisomicin; Sisomicin Sulfate; Sparfloxacin; Spectinomycin Hydrochloride; Spiramycin; Stallimycin Hydrochloride; Steffimycin; Streptomycin Sulfate; Streptonicozid; Sulfabenz; Sulfabenzamide; Sulfacetamide; Sulfacetamide Sodium; Sulfacytine; Sulfadiazine; Sulfadiazine Sodium; Sulfadoxine; Sulfalene; Sulfamerazine; Sulfameter; Sulfamethazine; Sulfamethizole; Sulfamethoxazole; Sulfamonomethoxine; Sulfamoxole; Sulfanilate Zinc; Sulfanitran; Sulfasalazine; Sulfasomizole; Sulfathiazole; Sulfazamet; Sulfisoxazole; Sulfisoxazole Acetyl; Sulfisboxazole Diolamine; Sulfomyxin; Sulopenem; Sultamricillin; Suncillin Sodium; Talampicillin Hydrochloride; Teicoplanin; Temafloxacin Hydrochloride; Temocillin; Tetracycline; Tetracycline Hydrochloride; Tetracycline Phosphate Complex; Tetroxoprim; Thiamphenicol; Thiphencillin Potassium; Ticarcillin Cresyl Sodium; Ticarcillin Disodium; Ticarcillin Monosodium; Ticlatone; Tiodonium Chloride; Tobramycin; Tobramycin Sulfate; Tosufloxacin; Trimethoprim; Trimethoprim Sulfate; Trisulfapyrimidines; Troleandomycin; Trospectomycin Sulfate; Tyrothricin; Vancomycin; Vancomycin Hydrochloride; Virginiamycin; Zorbamycin, or any combination thereof.

In further examples, the hydrophobic drug comprises ciprofloxacin. Ciprofloxacin is a quinolone antibiotic used to treat infections such as urinary tract infections, pneumonia, and skin and bone infections. Ciprofloxacin has the following chemical structure:

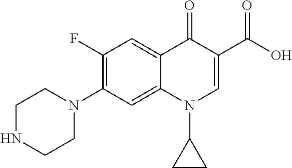 [SMILES: O=C(O)c1cn(C2CC2)c2cc(N3CCNCC3)c(F)cc2c1=O]

In some examples, there are at least two particles and the at least two particles comprise at least two different drugs, wherein each particle comprises one of the at least two different drugs. In further examples, each particle comprises a combination of the at least two different drugs.

The particles disclosed herein can be loaded with drugs and designed such that they are cell-specific, which makes treatment of a subject more effective and can also lessen symptoms to the subject, as well as other benefits. Furthermore, the cyclodextrins used to form the particles are derived from starches and are therefore biodegradable. The disclosed particles provide an advantage in the treatment of cancers because of their makeup, in that cancer cells utilize sugar and therefore because the cyclodextrin is a sugar, cancer cells will consume the particles comprising cancer treatment drugs, thereby treating the cancer with the drug of choice.

In further examples, the particle comprises an active agent. In certain examples, the active agent comprises a dye, a toxic compound, a phenolic compound, or any combination thereof.

An active agent is any substance, material, agent, or reagent that provides a desired effect when administered or utilized.

In further examples, the dye comprises methyl orange, methylene blue, methyl red, Evans blue, gentian violet, crystal violet, safranin, Eosin Y, fuchsine, acid fuchsin, carmine, acridine orange, or any combination thereof.

Dyes include, but are not limited to, acid dyes, natural dyes, basic (cationic) dyes, synthetic dyes, direct (substantive) dyes, disperse dyes, sulfur dyes, pigment dyes, mordant dyes, vat dyes, reactive dyes, macromolecular dyes, metallized dyes, naphthol dyes, premetallized dyes, gel dyeing, developed dyes, azo dyes, aniline dyes, and anthraquinone dyes.

In some examples, the toxic compound comprises fluorinated chemicals, phthalates, polychlorinated biphenyls, or any combination thereof.

Toxic compounds include any compound, chemical, or substance that upon exposure, cause harm to plants, animals, and/or humans. Toxic compounds can have a biological origin or a synthetic origin. In some examples, toxic compounds include mutagens, allergens, neurotoxins, and endocrine disruptors, for example.

In specific examples, the phenolic compound comprises curcumin, bisphenol A, chlorophenol, aminophenol, chlorocatechol, nitrophenol, methylphenol, or any combination thereof.

Phenolic compounds contain hydroxylated aromatic rings, the hydroxy group being attached directly to the phenyl, substituted phenyl, or other aryl groups. In some examples, phenolic compounds break through cellular membranes and in doing so, denature protein and lead to cell death and necrosis. Thus, containing these compounds presents a challenge that the particles disclosed herein overcome.

In specific examples, the particle has an average characteristic dimension of from 10 nm to 500 μm. In some examples, the particle has an average characteristic dimension of from 10 nm to 100 nm, 10 nm to 200 nm, 10 nm to 300 nm, 10 nm to 400 nm, 10 nm to 500 nm, 10 nm to 600 nm, 10 nm to 700 nm, 10 nm to 800 nm, 10 nm to 900 nm, 10 nm to 1 μm, 10 nm to 100 μm, 10 nm to 200 μm, 10 nm to 300 μm, or 10 nm to 400 μm. In further examples, the particle has an average characteristic dimension of from 100 nm to 200 nm, 200 nm to 300 nm, 300 nm to 400 nm, 400 nm to 500 nm, 500 nm to 600 nm, 600 nm to 700 nm, 700 nm to 800 nm, 800 nm to 900 nm, 900 nm to 1 μm, 1 μm to 100 μm, 100 μm to 200 μm, 200 μm to 300 μm, 300 μm to 400 μm, or 400 μm to 500 μm.

In specific examples, the particle has an average characteristic dimension of from 10 nm to 50 nm, 50 nm to 100 nm, 100 nm to 150 nm, 150 nm to 200 nm, 200 nm to 250 nm, 250 nm to 300 nm, 300 nm to 350 nm, 350 nm to 400 nm, 400 nm to 450 nm, 450 nm to 500 nm, 500 nm to 550 nm, 550 nm to 600 nm, 600 nm to 650 nm, 650 nm to 700 nm, 700 nm to 750 nm, 750 nm to 800 nm, 800 nm to 850 nm, 850 nm to 900 nm, 900 nm to 950 nm, 950 nm to 1 μm, 1 μm to 50 μm, 50 μm to 100 μm, 100 μm to 150 μm, 150 μm to 200 μm, 200 μm to 250 μm, 250 μm to 300 μm, 300 μm to 350 μm, 350 μm to 400 μm, 400 μm to 450 μm, or 450 μm to 500 μm. In certain examples, the particle has an average characteristic dimension of from 10 nm to 150 nm, 10 nm to 250 nm, 10 nm to 350 nm, 10 nm to 450 nm, 10 nm to 550 nm, 10 nm to 650 nm, 10 nm to 750 nm, 10 nm to 850 nm, 10 nm to 950 nm, 10 nm to 50 μm, 10 nm to 150 μm, 10 nm to 250 μm, 10 nm to 350 μm, or 10 nm to 450 μm.

In some examples, the particle has an average characteristic dimension of from 100 nm to 200 nm, 200 nm to 300 nm, 300 nm to 400 nm, 400 nm to 500 nm, 500 nm to 600 nm, 600 nm to 700 nm, 700 nm to 800 nm, 800 nm to 900 nm, or 900 nm to 1 pam.

In some examples, the particle comprises alpha-CD and beta-CD, beta-CD and gamma-CD, alpha-CD, and gamma-CD, or all three of alpha-CD, beta-CD, and gamma-CD.

In further examples, a particle comprises at least two drugs in one particle. In certain examples, a particle comprises at least three drugs in one particle.

### Cryogel

Further provided herein is a cryogel comprising the polymer disclosed herein. Cryogel, as used herein, refers to a supermacroporous gel networking developed by the cryogelation of monomers or polymeric precursors at subzero temperatures. Cryogels provide a highly porous material thereby allowing for the addition of drugs, and/or active agents, for example.

In further examples, the particle further comprises a therapeutically effective amount of a drug. In specific examples, the drug is hydrophobic. In certain examples, the hydrophobic drug comprises ciprofloxacin, nitrofurantoin, propranolol, a camphotosin analog, silbylin, docetaxel, doxorubicin, naproxen, trimethoprim/sulfamethoxazole, or any combination thereof.

In some examples, the cryogel further comprises an active agent. In further examples, the active agent comprises a dye, a toxic compound, a phenolic compound, or any combination thereof.

In specific examples, the phenolic compound comprises curcumin, bisphenol A, chlorophenol, aminophenol, chlorocatechol, nitrophenol, methylphenol, or any combination thereof.

In certain examples, the toxic compound comprises fluorinated chemicals, phthalates, polychlorinated biphenyls, or any combination thereof.

In some examples, the dye comprises methyl orange, methylene blue, methyl red, Evans blue, gentian violet, crystal violet, safranin, Eosin Y, fuchsine, acid fuchsin, carmine, acridine orange, or any combination thereof.

### Method

### Method of Administering a Drug

The present disclosure, in one aspect, provides for a method of administering a drug to a subject comprising administering to the subject the particle disclosed herein.

Also provided herein is a method of administering a drug to a subject comprising administering to the subject the cryogel disclosed herein.

In some examples, the drug is released slowly over a specified period of time. The release of a drug slowly over time is referred to as extended release. Extended release helps to maintain an appropriate concentration of the drug in the body, thereby providing a prolonged therapeutic effect.

### Method of Making a Particle

Provided herein is a method of making a particle comprising combining the at least one cyclodextrin and the cross linker. In some examples, combining the cyclodextrin and crosslinker includes polymerization. In further examples, polymerization includes emulsion polymerization, solution polymerization, suspension polymerization, and precipitation polymerization.

### Method of Making a Cryogel

Provided herein is a method of making a cryogel comprising combining the at least one cyclodextrin and the cross linker in a subzero temperature. This forms ice crystals which upon removal to room temperature, melt, resulting in the crosslinked network of the cryogel.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention, which are apparent to one skilled in the art.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

### Example 1: ESI-IM-MS Characterization of Cyclodextrin Complexes and their Chemically Cross-Linked Alpha (α-), Beta (β-) and Gamma (γ-) Cyclodextrin Particles as Promising Drug Delivery Materials with Improved Bioavailability

### INTRODUCTION

Cyclodextrins (CDs) are natural cyclic oligosaccharides with a relatively hydrophobic cavity and a hydrophilic outer surface. Herein, alpha (α-), beta (β-) and gamma (α-) CD particles were prepared by directly using α-, β-, and γ-CDs as monomeric units and divinyl sulfone (DVS) as a crosslinker in a single-step via reverse micelle microemulsion crosslinking technique. Particles of p(α-CD), p(β-CD), and p(γ-CD) were perfectly spherical in sub- 10 μm size ranges. The prepared p(CD) particles at 1.0 mg/mL concentrations were found biocompatible with >95% cell viability against L929 fibroblasts. Furthermore, p((α-CD) and p(β-CD) particles were found non-hemolytic with <2% hemolysis ratios, whereas p(γ-CD) particles were found to be slightly hemolytic with its 2.1±0.4% hemolysis ratio at 1.0 mg/mL concentration. Furthermore, a toxic compound, Bisphenol A (BPA) and a highly antioxidant polyphenol, curcumin (CUR) complexation with α-, β-, and γ-CD molecules was investigated via Electrospray-Ion Mobility-Mass Spectrometry (ESI-IM-MS) and tandem mass spectrometry (MS/MS) analysis. It was determined that the most stable noncovalent complex was in the case of R-CD, but the complex stoichiometry was changed by the hydrophobic nature of the guest molecules. In addition, BPA and CUR were separately loaded into prepared p(CD) particles as active agents. The drug loading and release studies showed that p(CD) particles possess governable loading and releasing profiles.

Cyclodextrins (CDs) are natural cyclic oligosaccharides of sugar consisting of D-glucose units linked to each other in different numbers by α-1, 4 glycosidic bonds. Pharmaceutically important CDs are called alpha (α-), beta (β-) and gamma (γ-) cyclodextrins containing 6, 7 and 8 glycopyranose units, respectively.

The toroidal special arrangement in three dimensions of CDs results in truncated cone-like structures with a relatively hydrophobic inner part composed of nonpolar structures owing to CH groups (C3 and C5) and glycosidic oxygen atoms and a hydrophilic outer periphery owing to the hydroxyl groups. The relatively hydrophobic interior zone is able to accommodate lipophilic and hydrophobic molecules through non-covalent interactions. These interactions also called host-guest inclusion complexes that are formed via the mechanism that the less polar part of the guest molecule enters the hydrophobic cavity and the more polar and/or mostly charged group is directed towards the solvent, outside the wider part of the cavity. As the reversible interaction of sterically compatible nonpolar parts of the lipophilic molecules within the hydrophobic cavity of CDs render spontaneous supramolecular complex formation in an aqueous environment, and the hydrophobic drugs become water-soluble due to the presence of hydroxyl groups in outer regions of CDs. This distinctive ecosystem of CDs makes them exceptional materials as specific molecule carriers. As α-, β, and γ-cyclodextrins have different molecular sizes and cavity diameters, they can engulf suitable different molecules for delivering them into different environments including a wide variety of drug molecules with different physicochemical properties. Furthermore, the precise size of CDs renders them the ability for specific adsorption of matching molecules such as pesticides, dyes, drugs, and certain metal ions some of which are known as toxic carcinogenic compounds that need to be selectively separated from drinking waters, paints, textile, food and packing industry.

Upon the examination of cyclodextrins separately, α-CDs have 6 glucopyranose units with a central cavity diameter of 4.7-5.3 A°. These native cyclodextrins are moderately soluble in water, tasteless, odorless, stable thermally stable in alkaline or acid solutions (up to 200° C.). Therefore, α-CDs are frequently used in various fields such as nutrition, health, textile, food industry, and pharmaceutical technology. For example, α-CDs are resistant to the hydrolysis of human saliva and pancreatic amylases thus they are used as carriers and stabilizers for sweeteners and emulsifiers in food technology. In addition, α-CDs can bind preferentially saturated and trans fatty acids and their high specificity for “bad” fatty acids makes them particularly interesting as dietary fibers.

Beta cyclodextrins (β-CDs) have 7 glucopyranose units with an internal cavity diameter of 6.0-6.5 A° so that they can fit larger biomolecules in comparison to α-CD. Also, β-CDs have various advantages such as non-toxicity, low cost, higher yield, parenteral safety, bio-tolerability, and protection against premature degradation in biological systems. Moreover, β-CD has higher complexing ability with crosslinkers, compared to other natural CDs. The molecular dimensions of the R-CD cavity make it the most ideal host among the three natural CDs for complex formation. To date, β-CDs have been used for solubilizing various hydrophobic compounds such as synthetic steroids, certain polyphenolics, and adsorbents for pollutants such as toxic dyes, Bisphenol A, a carcinogenic compound, pesticides and so on. Although they have favorable properties, the solubility of β-CDs is quite low due to high lattice energy and internal hydrogen bond network, which raise an important problem in their use.

Lastly, gamma cyclodextrins (γ-CDs) have 8 glucopyranose units with a central cavity diameter of 7.5-8.3 A° and because of this larger interior space, γ-CDs can carry larger molecules in comparison to other CDs. In addition, γ-CDs have higher water solubility and better bioavailability than other native CDs and this main property can offer more inducements in the stabilization of bigger guest molecules against oxidation, increased drug permeability through biological membranes, high therapeutic index, and well-tolerated drug delivery profile. Because of these features, γ-CDs have been used in various fields such as selective CO₂ adsorption, removal of hazardous materials, parenteral formulations and in vitro drug delivery. However, the use of γ-CD is considerably limited due to its low efficiency and high price.

CDs improved the solubility, bioavailability, and physical and chemical stability of certain drugs. The low solubility barriers of native CDs can be overcome by modifying CDs. CDs can self-assemble, cross-link, polymerize/co-polymerize with other compounds, and their complexation ability is often preserved or even enhanced. Therefore, CDs can be improved to gain additional features e.g., easy penetration, long-term physical and microbiological stability, improved solubility, larger surface area, ability to carry multiple payloads, high drug loading capacity and controlled drug release. Various crosslinkers such as epoxides, polyacrylic esters, polycarbonate acids, polyamidoimines, tetrafluoroterephthalonitrile (TFTN), and divinyl sulfone (DVS) used to prepare CD-based micro/nanoparticles. Various interactions such as Van der Waals, hydrophobic, hydrogen bonds, electrostatic interactions and steric strain play a role in the functionalization of natural CDs, resulting in supramolecular CD-based material formation with advanced properties.

Bisphenol A (BPA) is a synthetic compound mainly used in the synthesis of polycarbonates and epoxy resins. BPA appears in many products in daily life such as electronic equipment, paper, food-packaging materials, medical devices, dental products, water pipes and bottles which are quite common for everyday use. In particular, food and drinking water is the most important source of BPA exposure. Other exposure pathways to BPA are inhalation and dermal exposure and humans can readily get affected because BPA is a quickly metabolized substance. High exposure to BPA is found to be correlated with imbalanced reproductive hormone levels, insulin resistance, corrupted thyroxin metabolism, neurotoxicity and disrupted endocrine functions. BPA also has mutagenic and carcinogenic effects, which cause safety concerns, especially for infants and pregnant women.

Curcumin (CUR), mainly known as a coloring agent and food additive, is a natural polyphenolic compound used in traditional remedies. CUR has unique curative and protective effects such as wound healing, anti-inflammatory, antioxidant, anti-proliferative, anti-atherosclerosis, neuroprotective and anti-tumor activities. Despite all these special properties, curcumin's applications are quite limited because of its hydrophobicity and poor oral bioavailability, low cellular uptake, rapid metabolism and susceptibility to chemical degradation.

As α-, β- and γ-CDs have structures that contain both hydrophilic and hydrophobic properties innately, and upon modification, these compounds can even become custom-made ideal carriers for specific drug molecules. Herein, single-step preparation of divinyl sulfone (DVS) cross-linked poly(α-CD) (p(α-CD)), poly(β-CD) (p(β-CD)) and poly(γ-CD) (p(γ-CD)) micro-/nanoparticles via simultaneous polymerization/cross-linking mechanism in reverse micelle microemulsion with spherical geometries was reported. The morphologic properties of prepared particles were examined by scanning electron microscopy (SEM), Fourier transforms infrared (FT-IR) spectroscopy, and zeta potential (ZP) analyzer. Moreover, the thermal stability of prepared materials was characterized via thermogravimetric analysis (TGA). Furthermore, the biological properties of prepared CD-based materials such as hemo- and cell compatibility were evaluated, as these characteristics are very important for their potential use in certain biomedical applications. Moreover, the use of these p(CD) particles as drug delivery vehicles was examined using BPA or CUR as active agents.

### Materials and Methods

### Materials

Cyclodextrins, α-cyclodextrin (α-CD, 98%, spectrum chemical MFG CORP), β-cyclodextrin (β-CD, minimum 98%, Sigma), γ-cyclodextrin (γ-CD, <%99, TCI), and the crosslinker divinyl sulfone (DVS, 97%, Merck) were used as received. Dioctyl sulfosuccinate sodium salt (96%, Acros Organics) as a surfactant, 2,4-trimethylpentane (isooctane, ≥99.5%, Isolab), and acetone (99%, BRK) as solvents were used in the preparation of cyclodextrin particles. Methanol and formic acid (LC-MS Grade) were purchased from Sigma-Aldrich (St Louis, MO, USA). ESI-L Low Concentration Tuning Mix was purchased from Agilent Technologies (Santa Clara, CA, USA).

The L929 fibroblast cell line was obtained from the SAP Institute (Ankara, Turkey). Dulbecco's Modified Eagle's Medium (DMEM containing 4.5 g/L glucose, 3.7 g/L sodium pyruvate) supplemented with L-Glutamine and fetal bovine serum (FBS, Pan Biotech, Aidenbach, Germany) was purchased from Pan Biotech (Aidenbach, Germany) and used as the cell growth medium. Trypsin (0.25%) was purchased from Pan Biotech (Aidenbach, Germany) and used for detaching the adherent L929 cells. Dimethyl sulfoxide (DMSO, 99.9%, Carlo Erba, France) and colorimetric agent 3-(4,5-dimethylthiazol-2-yl)-2,5-diphenyltetrazolium bromide (MTT, neofroxx, Germany) were used as received. Bisphenol A (BPA, Sigma-Aldrich, 97%) and Curcumin (CUR, ≥94%, Sigma-Aldrich) were used as received. Ultra-pure deionized water (DI water, resistivity of 18.2 M·Ω·cm) was obtained from a Millipore Direct-Q 3 UV water purification system used for the preparation of all aqueous solutions.

Preparation of poly(α-Cyclodextrin (p(α-CD)) and poly(γ-cyclodextrin) (p(γ-CD)) Particles

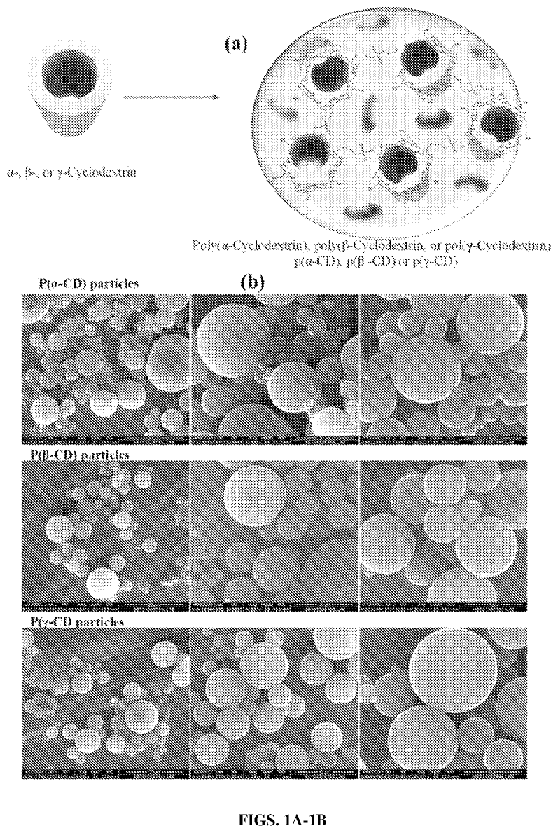

The particles of α-CD, γ-CD were prepared by following the same procedure reported for the preparation of p(β-CD) in the literature [7]. For this purpose, 0.5 g of α-CD or γ-CD were dissolved in 5 mL 0.25 M NaOH solution. Next, 0.5 mL of 0.1 g/mL cyclodextrin oligomers solutions in 0.25 M NaOH were placed into 30 mL of 0.2 M Bis(2-ethylhexyl) sulfosuccinate sodium salt (AOT) in isooctane under continuous stirring at 1000 rpm and room temperature. Then, the crosslinker, divinyl sulfone (DVS) which 100% based on the repeating unit hydroxyl units (6 for α-CD, and 8 for γ-CD), e.g., 18 μL for α-CD was separately added into microemulsions at a 100% ratio with respect to the stoichiometric mole ratio of C6 hydroxyl groups on α-CD. The emulsions were stirred for 2 h at room temperature, and particles of p(α-CD) and p(γ-CD) were collected by precipitation in an excess amount of acetone. Then, the obtained particles were washed via centrifugation at 10 000 rpm few times using the following solvents: acetone (thrice), acetone/ethanol (50:50 VN, two times), ethanol, ethanol/water (50:50 VN, twice), and water (twice) to remove unreacted reagents and surfactants. Finally, the purified p(α-CD) and p(γ-CD) particles were dried via a freeze-dryer and capped in airtight containers until further usage. The same synthesis procedure was employed for the preparation of co-polymeric and terpolymeric forms of α-, -, γ-CDs particle as p(α-CD-co-β-CD), p(α-CD-co-γ-CD), and p(β-CD-co-γ-CD) and p(α-CD-co-β-CD-co-γ-CD) containing desired amounts of α-, β-, γ-CDs. Then, these prepared copolymeric and terpolymeric forms α-, β-, γ-CD particle were washed via centrifugation as mentioned above. The SEM images of p(α-CD), p(β-CD) and p(γ-CD) are given in FIG. 1A and FIG. 1B. As can be seen the particle size of p(α-CD), p(β-CD) and p(γ-CD) are in few hundred nanometers to few tens of micrometer.

Preparation of p(α-CD), p(fi-CD) and (p(γ-CD) cryogels

Cryogels of α-, β-, γ-CDs were synthesized by following literature. The synthesis of supe porous of a, p, γ-CDs based cryogels were carried out via cryo-crosslinking technique. Initially, 0.5 g of α-, β-, γ-CDs oligosaccharides was weighed and placed into 5 mL of 0.25 M NaOH solution separately in a vial. After dissolving these CDs, the vial was placed into deep freezer at −20° C. for 3 min to cool down the solution. Then, the crosslinker, DVS was separately added into solutions at 200% mole ratios with respect to stoichiometric mole ratios of the numbers of hydroxyl groups on α-CD, and γ-CDs. For example, 360 μL of the cross-linker, divinyl sulfone (DVS) based on the repeating unit hydroxyl units (6 for α-CD, and 8 for γ-CD), on C6 hydroxyl groups of α-CD and γ-CD cyclodextrins, respectively as 200% ratio was added to CDs solutions. After vortex mixing 1 min, the cryogels precursors were transferred into plastic straws (6 mm in diameter) and placed into deep-freezer at −20° C. for 24 h for cryo-crosslinking. The prepared super porous cryogels of p(α-CD), p(β-CD), and p(γ-CD) were cut in similar cylindrical shapes and sizes, ˜1 cm, and were washed with excess amounts of distilled water several times in a beaker to remove unreacted molecules and the excess amount of NaOH from the structure. The wash water was changed every 6 h during 24 h washing time. The cleaned super porous p(α-CD), p(β-CD) and p(γ-CD) cryogels were then dried via a freeze-dryer and capped in air-tight containers until further usage.

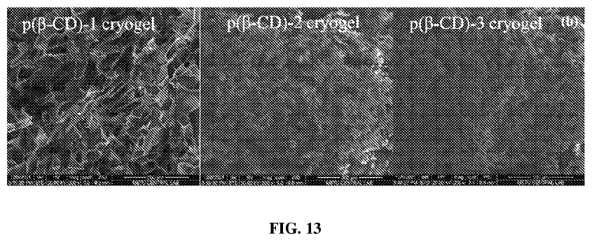

The same synthesis procedure was employed for the preparation of co-polymeric and terpolymeric forms of α-, β-, γ-CDs cryogels as p(α-CD-co-β-CD), p(α-CD-co-γ-CD), and p(β-CD-co-γ-CD) and p(α-CD-co-β-CD-co-γ-CD) containing desired amounts of α-, β-, γ-CDs. Then, these prepared co-polymeric and terpolymeric forms α-, β-, γ-CDs particle were washed in excess amount of distilled water as mentioned above. The scanning electron microscopy images of p(β-CD) cryogels are given in FIG. 13. As can be seen, the pore size of p(β-CD) is from a few hundred nanometers to few tens of micrometer.

Preparation of poly(α-Cyclodextrin) (p(α-CD)), poly(β-Cyclodextrin (p(β-CD)) and poly(γ-cyclodextrin) (p(γ-CD)) Particles

The particles of α-CD, β-CD and γ-CDs were prepared. For this purpose, 0.5 g of α-CD or γ-CD was dissolved in 5 mL of 0.25 M NaOH solution. Next, 0.5 mL of 0.1 g/mL cyclodextrin monomer solutions in 0.25 M NaOH were placed into 30 mL of 0.2 M Bis(2-ethylhexyl) sulfosuccinate sodium salt in isooctane under constant stirring at 1200 rpm and room temperature. Then, the cross-linker, divinyl sulfone (DVS) 100% based on the hydroxyl units of CD molecules (6 for α-CD, 7 for β-CD and 8 for γ-CD) was added into each reaction medium. After stirring the emulsions for 2 h, the particle formation was completed. Following this, particle-containing medium was centrifuged at 10 000 rpm. After this, particles of p(α-CD), p(β-CD) and p(γ-CD), then, washed via centrifugation at 10 000 rpm using the following process: acetone (twice), ethanol/acetone (20:30 V/V, twice), ethanol, ethanol/water (20:30 V/V, once), and DI water to discard unreacted reagents. Lastly, the cleansed particles were dried by a freeze-dryer.

Characterization of p(α-CD), p(β-CD), and p(γ-CD) Particles

For SEM analysis, dry CD-based particles were covered with palladium/gold to a few nm under a vacuum for 10 seconds and the images were acquired by SEM (Quanta 400F field emission SEM) with a 10 kV operating voltage. Fourier Transform Infrared Radiation (FT-IR, Perkin-Elmer, Spectrum 100, US) spectra of CD-based materials was recorded in the frequency range of 4000 to 650 cm⁻¹ with 4 cm⁻¹ resolutions by using a FT-IR spectrophotometer. Thermal degradation of CD-based materials was determined by using thermogravimetric analysis (TGA, SII TG/DTA 6300, Japan) under N₂ atmosphere at 2 mL/min flow rate with 10° C./min heating rate from 50 to 700° C. Zeta potentials (ZP) values of p(α-CD), p(β-CD), and p(γ-CD) were measured by using a zeta potential analyzer (Malvern Zetasizer, Nano Z S series, Brookhaven Inc., NY, USA) at 25° C. The pH-dependent change in the surface charges of CD-based particles was analyzed in the pH 1-12 range. The ZP measurements of CD-based particles were done on 1 mg/mL concentration of the particles in triplicates.

Blood Compatibility of p(α-CD), p(β-CD), and p(γ-CD) Particles

Hemolysis and blood clotting assays were performed to investigate the blood compatibility of linear α-CD, β-CD, and γ-CD and their particle forms. Human blood was obtained from healthy volunteers and approved by the Clinical Research Ethics Committee of Canakkale Onsekiz Mart University (2011-KAEK-27/2022-2200063689) and placed into tubes containing EDTA. Before the analysis, all solutions were preheated to 37° C.

For the hemolysis assay, diluted blood was prepared by using 1:1.25 (v:v) ratio of blood:0.9% aqueous NaCl solution and 200 μL of the diluted blood was interacted with linear CD solution and CD particle suspensions in 10 mL of 0.9% saline solution at 1 mg/mL concentration in a water bath at 37° C. for 1 h. In the separation tubes, 200 L of the diluted blood was added into 10 mL of 0.9% aqueous NaCl solution and DI water as a negative and positive control, respectively. Then, the tubes were centrifuged at 100 g for five minutes and the absorbance values for the supernatants were measured at 542 nm with UV-Vis spectroscopy (T80+UV/VIS spectrometer, PG Instrument Ltd. Leicestershire, UK). The hemolysis ratio of the linear CD or CD particles was evaluated using the following equation.

$$Hemolysis ratio \% = \frac{( {A}_{material} - {A}_{negative} )}{( {A}_{positive} - {A}_{negative} )} \times 100$$

Where Aₘₐₜₑᵣᵢₐₗ is the absorbance value of the blood solution interacted with materials in 0.9% aqueous NaCl solution. A_{negative} and Aₚₒₛᵢₜᵢᵥₑ are the absorbance values of the blood solution without materials in 0.9% aqueous NaCl solution and in DI water, respectively. All assays were carried out in triplicate and the results are given with standard deviations.

For the blood clotting assay, 64 μL of 0.2 M CaCl₂ aqueous solution was mixed with 810 μL of blood containing EDTA and immediately 270 μL of this blood was covered with 10 mg of linear CD or CD particle. After 10 min, 10 mL of DI water was slowly added into the tubes and centrifuged at 100 g for 1 minute. Then 10 mL of supernatant solution containing non-clotting blood was taken from the tube and diluted with 40 mL of DI water. In the separation tube, 250 μL of the blood containing EDTA was dispersed in 50 mL of DI water as a control. The blood solution was incubated at 37.5° C. in a water bath for 1 h and then, the absorbance value of the supernatant was measured at 542 nm by using UV-Vis spectroscopy. The blood clotting index of the linear CD or CD particles was evaluated from the following equation.

$$Blood Clotting Index = ( {A}_{material} / {A}_{control} ) \times 100$$

Where Aₘₐₜₑᵣᵢₐₗ is the absorbance value of the blood solution interacted with the CD-based materials and A_{contol} is the absorbance value of the blood solution without the CD-based materials as a control. All assays were carried out in triplicate and the results are given with standard deviations.

Cytotoxicity analysis of p(α-CD), p(β-CD), and p(γ-CD) particles

For the biocompatibility analysis, L929 fibroblasts were cultured in an air-humidified incubator (5% CO₂ atmosphere) in DMEM supplemented with 10% fetal bovine serum (FBS) at up to 70% cell confluency. The cytotoxicity analysis of CD-based materials was performed by MTT assay which determines the metabolic activity of living cells.

The L929 fibroblast cell suspension was adjusted to a density of 5×10⁵ cells mL⁻¹ and gently homogenized. Then, 100 L of this cell suspension was seeded onto wells on a 96-well plate. The well-plate was incubated at 37° C. under 5% CO₂ in an air-humidified incubator for 24 h. After adherence of the fibroblasts, the culture medium was removed from the wells and 100 μL of CD-based particle suspension in culture medium DMEM at different concentrations between 50 and 1000 μg/mL was added to the cells. The plate was incubated at 37° C. under 5% CO₂ in an air-humidified incubator for 24 h more. At the end of the incubation, the medium was removed from the wells, the cells were washed with phosphate-buffered saline solution (PBS) two times. After this step, 100 μL of MTT (Tetrazolium dye) at 0.5 mg/mL concentration was added to each well. The plate was incubated at room temperature in the dark for 4 h. Then, MTT solution was removed from the wells and 200 μL of DMSO was placed into the wells to dissolve the blue formazan crystals. Finally, the absorbance values were measured at 590 nm with a plate reader (Thermo Scientific™ Multiskan™). The absorbance value of control group was accepted as 100% viability and the decrease in cell viability % was estimated based on the absorbance values of the wells treated with CD-based materials by the following equation, Cell viability

$$Cell viability \% = ( {A}_{material} / {A}_{control} ) \times 100$$

Where Aₘₐₜₑᵣᵢₐₗ is the absorbance value of the cells interacting with CD-based materials and A_{control} is the absorbance value of the untreated cells as control. All assays were carried out in triplicate and the results are given with standard deviations.

### Electrospray-Ion Mobility-Mass Spectrometry (ESI-IM-MS) and Tandem Mass Spectrometry (MS/MS) Analysis of CD-BPA or CUR

Stock solutions of CD/BPA or CUR were prepared in water/ethanol at a concentration of 1.0×10⁻² M. The mixture solutions were prepared with stock solutions in a molar ratio of 1:1 (CD/BPA or CUR) and they were incubated at room temperature for 2 h. All mixture solutions were diluted with water: methanol: formic acid (30:70:0.1, v/v) to a final concentration of 1.0×10⁻⁴ M.

ESI-IM-MS and MS/MS analyses of CD/BPA or CUR were performed on a trapped ion mobility spectrometry-time-of-flight mass spectrometer (TIMS-TOF-MS) instrument (Bruker Daltonics, Bremen) with an electrospray ionization (ESI) source. The mixture solutions were infused via a syringe pump at a rate of 5 μL/min. The instrument was operated in a negative mode for all MS analyses. In IM-MS analysis, the ESI source was operated at 500 V end plate offset, 3600 V capillary voltage, 0.4 bar nebulizer gas, 4 L/min drying gas flow, and 200° C. drying gas temperature. The TIMS mode settings were ramp time start 0.42 Vs/cm², ramp time end 1.94 Vs/cm², ramp time 350 ms, and tunnel in-tunnel out pressure difference was set to 1.8 mbar. Nitrogen was used as the collision gas for all MS/MS analyses and collision energies 2-30 eV (E_{lab}) for CDs/BPA, 3-45 eV (E_{lab}) for CDs/Curcumin complexes. The source operating conditions for MS/MS analysis were 500 V end plate offset, 3000 V capillary voltage, 0.4 bar nebulizer gas, 4 L/min drying gas flow, and 200 C drying gas temperature. Agilent ESI-L Low Concentration Tuning Mix standard used for Daily mass and ion mobility calibration. All the obtained data were processed with Data Analysis 5.0. software (Bruker Daltonics).

MS/MS analyses were performed at different collision energy (CE) values to obtain the survival yield (SY) of the precursor ions. SY values were calculated for each precursor ion from Equation 1.

$$SY = {I}_{P} / ( {I}_{p} + \sum {I}_{F} )$$

Where Iₚ is the intensity of the precursor ions and IF is the intensity of fragment ions.

Center-of-Mass energies were calculated using Equation 2,

$${E}_{CM} = ( {m}_{g} / {m}_{g} + {m}_{p} ) \times {E}_{lab}$$

Where E_{lab} is the laboratory energy (CE), m_{g} and mₚ are the mass of the neutral gas and precursor ion, respectively.

Bisphenol a or Curcumin Loading into p(α-CD), p(β-CD), and p(γ-CD) Particles

Bisphenol A (BPA) and curcumin (CUR) as active agents were loaded into p(α-CD), p(β-CD), and p(γ-CD) particles using the adsorption technique. In the loading process, the exact amount of BPA, a 2-fold mole ratio of α-, β-, or γ-CD molecules in CD-based particles, was dissolved in 5 mL of ethanol for 5 min and 35 mL of DI water was added into this solution. P(α-CD), p(β-CD), and p(γ-CD) particles weighing 100 mg each were added into BPA solution and stirred at room temperature for 48 h. Then, BPA-loaded CD-based particles were washed with ethanol-water (1:1) mixture one time by centrifugation at 10 000 rpm for 10 min. The BPA loaded CD-based particles were dried by freeze dryer for 48 h.

Similarly, curcumin (CUR) was loaded into p(α-CD), p(β-CD), and p(γ-CD) particles by using the same procedure as described above. Briefly, a certain amount of CUR, a 2-fold mole ratio of CD molecules within CD-based particles, was dissolved in 30 mL of ethanol for 10 min and 20 mL DI water was added into this solution. P(α-CD), p(β-CD), and p(γ-CD) particles weighing 100 mg were added into CUR solution and stirred at room temperature for 48 h. Then, p(α-CD), p(β-CD), and p(γ-CD) particles were washed with ethanol-water (1:1) mixture one time by centrifugation at 10 000 rpm for 10 min. The CUR-loaded p(CD) materials were dried by freeze dryer for 48 h.

The absorbance value of the BPA/CUR solutions before and after the loading processes were measured by using UV-Vis spectroscopy (SP-UV300SRB, Spectrum, China) at 275 nm and 425 nm for BPA and CUR, respectively against the previously constructed corresponding calibration curves for BPA and CUR prepared in ethanol-water mixtures. The ethanol-water solution was prepared at 1:7 and 3:2 volume ratios for BPA and CUR, respectively.

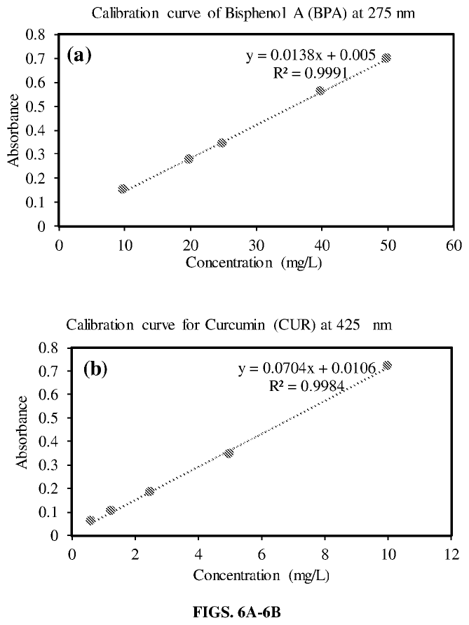

The calibration curves of BPA at 275 nm wavelength and CUR at 425 nm wavelength were given in FIG. 6A -FIG. 6B.

In Vitro Drug Release Profile of Bisphenol a (BPA) and Curcumin (CUR) from p(α-CD), p(β-CD), and p(γ-CD) Particles

Drug release studies of BPA or CUR were performed both in ethanol and PBS solutions, separately. Briefly, 50 mg of BPA-loaded p(α-CD), p(β-CD), and p(γ-CD) particles were dispersed in 1 mL of ethanol or PBS and transferred to a dialysis membrane. These particles containing membranes were placed into 20 mL of ethanol or PBS solution at 37° C. in a shaker bath. The drug-releasing mediums were then sampled and evaluated using a UV-Vis spectrometer at 275 nm to determine the amount of BPA against the previously generated calibration curves for BPA prepared in ethanol or PBS and the released amounts of drugs were calculated. The analysis was repeated three times, and the values are reported as the average values with standard deviations.

Similarly, drug release studies of CUR were performed in ethanol or PBS solutions. First, 50 mg of CUR-loaded p(α-CD), p(β-CD), and p(γ-CD) particles were dispersed in 1 mL of ethanol or PBS solution and transferred to a dialysis membrane. The particle containing membranes were then placed into 20 mL of ethanol or PBS solution at 37° C. in a shaker bath. The drug releasing mediums were sampled and evaluated by UV-Vis spectrometer at 425 nm to determine the amount of CUR via the previously prepared calibration curves for CUR in ethanol or PBS, and the released amounts of drugs were calculated. The analysis was repeated three times, and the values are reported as the average values with standard deviations.

### Results and Discussion

Particles of α-CD, β-CD, and γ-CD particles as p(α-CD), p(β-CD), and p(γ-CD) were prepared by crosslinking of α-CD, β-CD, and γ-CD with DVS crosslinker at 100% mole ratio of CD molecule via Michael addition reaction. As illustrated in FIG. 1A, the α-CD, β-CD, and γ-CD unit can be chemically linked to attain the corresponding particles with the reaction of the crosslinker, divinyl sulfone (DVS) and their SEM images as shown in FIG. 1B demonstrated that the particles are precisely spherical, their sizes are in the range of few hundred nanometers to tens of micrometers.

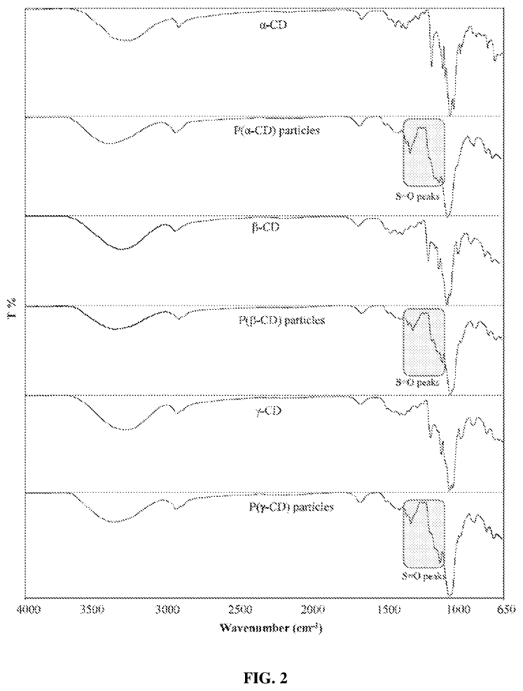

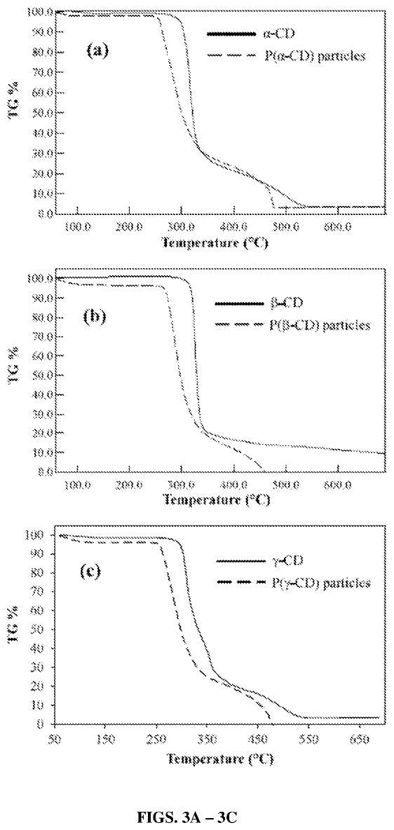

The chemical functional groups of α-CD, β-CD, and γ-CD and their crosslinked particle forms were corroborated by FT-IR analysis and the corresponding spectra are presented in FIG. 2. Thermal degradation profiles of α-CD, p(α-CD) particles, β-CD, p(β-CD) particles, γ-CD, and p(γ-CD) particles were illustrated in FIG. 3A-FIG. 3C, respectively.

The characteristic peaks of the CD unit were affirmed at 3600-3100 cm⁻¹ attributed to hydroxyl stretching, and the peak at 2930 cm⁻¹ due to symmetric and asymmetric CH₂ stretching vibrations, and the peaks in 1300-1400 cm⁻¹ range ascribed to C—H stretching vibrations, and the peaks at 1030 cm⁻¹ attributed to C-O-C vibrations. Peaks at 1310 cm⁻ and 1280 cm⁻ were defined as the main peaks for S═O stretching vibrations.

The crosslinking reaction between hydroxyl groups of CD and vinyl groups from DVS could be confirmed because of the specific S═O stretching peaks belonging to DVS for all particles. These results uphold that p(α-CD), p(β-CD), and p(γ-CD) particles were successfully synthesized using an equal mole of DVS (100%) with α-CD, β-CD, and γ-CD units in a single step.

In FIG. 3A, the first main degradation of α-CD is in 300° C. to 350° C. with 72.7% weight loss, and the second slight degradation step was measured in 350-550° C. range with 93.4% weight loss. P(α-CD) particles were determined to be slightly more thermally degradable than α-CD with two main degradation steps in the 246-350° C. range with 71.1% weight loss and in 400-492° C. with 97.2% weight loss. FIG. 3B revealed that β-CD was degraded only with a single main step in 312-351° C. with 79.6% weight loss and degradation was slightly continued up to 689° C. with 90.5% weight loss, whereas the first main degradation of p(β-CD) particles was seen in 262-343° C. range with 80.1% weight loss and completely degraded at 459° C. Also, γ-CD was shown two main degradation steps one of which is in the 340-360° C. range with 80% weight loss, and the second one in is 450-545° C. with 96.6% weight loss value. P(γ-CD) particles were found more thermally degradable with two main degradation steps which are in the 255-350° C. range with 75% weight loss and in 400-478° C. with almost complete weight loss as illustrated in FIG. 3C.

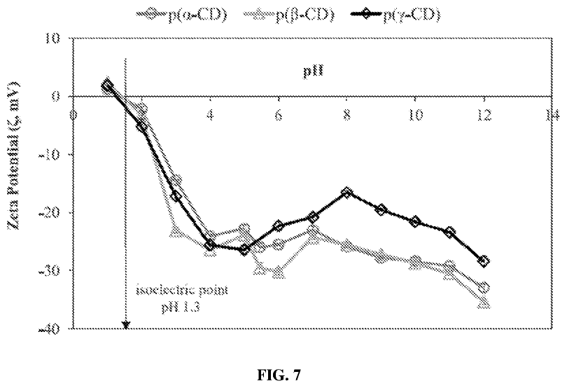

The surface charge of materials provides valuable information regarding the surface properties of p(CD) particles and the magnitude of zeta potential determines to assess the potential use of these particles in various fields including biomedical and pharmaceutical applications. Therefore, the change in surface charges of DVS crosslinked p(α-CD), p(β-CD), and p(γ-CD) particles was examined in pH 1-12 range, and the zeta potential vs pH graphs was plotted results are shown in FIG. 7.

The pH of p(α-CD), p(β-CD), and p(γ-CD) particles solutions at 1 mg/mL concentration were evaluated as 5.45 in DI water, and their surface charges were found as −26.0±0.8 mV, −29.5±0.8 mV, −22.3±0.8 mV, respectively. It is worthy to note that p(α-CD), p(β-CD), and p(γ-CD) particles possess negative surface charges at neutral pH, 7 as −23.0±0.6 mV, −24.3±0.2 mV, and −20.8±0.4 mV, respectively. Therefore, all three CD-based particles are expected to be colloidally stable at physiologic pH. The isoelectronic point of the p(α-CD), p(β-CD), and p(γ-CD) particles where they attain zero net surface charge were determined about pH 1.3, from the graph given in FIG. 7. Isoelectric point of pH 1.1-1.2 was reported for p(β-CD) particles crosslinked with DVS is consistent with the current results.

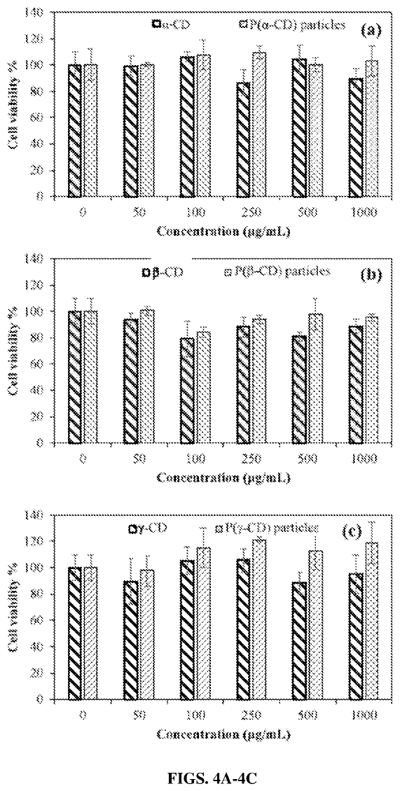

To assess the toxicity of the particles, cell viability tests of p(α-CD), p(β-CD), and p(γ-CD) particles were performed on L929 fibroblasts at concentrations ranging from 50 to 1000 μg/mL and time as the results are summarized in FIG. 4A-FIG. 4C, respectively.

The cell viability against L929 fibroblasts in the presence of α-CD molecules and p(α-CD) particles, β-CD molecules and p(β-CD) particles, γ-CD molecules and p(γ-CD) particles at 1.0 mg/mL concentration were determined as 90±7% and 103±5%, 89±14% and 95±3%, and 95±14%, and 119±15%, respectively. Therefore, it is apparent that all crosslinked particle forms of CDs show good biocompatibility for fibroblast cells with almost more than 100% cell viability even at 1.0 mg/mL concentrations. This attests that the particles of CD regardless of their types whether they are derived from α-, β-, or γ-molecules are biocompatible and can safely be used in cell-contacting biomedical applications.

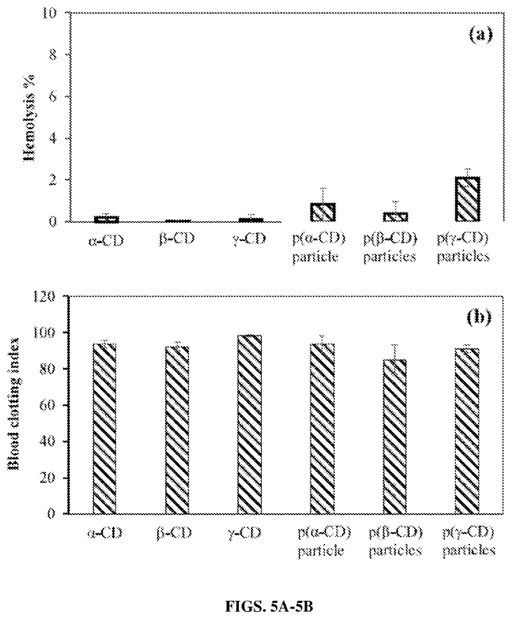

The hemocompatibility of CD molecules and particles at 1 mg/mL concentrations was investigated via hemolysis and blood clotting index assays and the corresponding results are given in FIG. 5A-FIG. 5B, respectively.

Except for p(γ-CD) particles, linear and particle forms of all CDs were found non-hemolytic materials with <2% hemolysis ratios, but γ-CD particles were determined as very slightly hemolytic material with its 2.1±0.4% hemolysis ratio at 1.0 mg/mL concentration. It is known that materials with <2% hemolysis ratios are considered blood compatible whereas materials with >2% hemolysis ratios are non-hemolytic. Furthermore, blood clotting indexes of α-CD, β-CD, γ-CD molecules as 93.6±2.2%, 92.3±2.3%, 98.4±0.7%, and their particle forms as 93.8±4.7%, 85.2±8.1%, and 90.9±2.0% were determined, respectively at 1 mg/mL concentration. The particle forms of CDs asserted high blood clotting indexes. These results supported that all types of CD-based particles possessing non-hemolysis behavior with blood compatibility ascertain their safe blood contacting applications, e.g., intravenous applications up to 1 mg/mL concentrations.

To evaluate the complex stability and geometry of the studied host-guest complex ability of α-, p3- and γ-cyclodextrin with BPA and CUR in gas-phase, ESI-IM-MS and MS/MS techniques were used. First collision cross-section values of CDs, BPA and CUR were obtained by ESI-IM-MS analysis and then collision cross section values of host-guest complexes were carried out. All collision cross section values representing approximately gas-phase shape of the species are given in Table 1.

TABLE 1
Overview of the observed BPA, CUR, CDs, and CD complexes
with experimental (Exp) mass and collision cross section.

| | | Measured | Measured | CCS_{(Exp)} Difference |
|---|---|---|---|---|
| | | Mass_{(Exp)} | CCS_{(Exp)} | Between Complex and |
| Samples | z | (m/z) | (Å²) | Cyclodextrin (Å²) |
| [BPA-H]⁻ | 1 | 227.98 | 160.8 | — |
| [CUR-H]⁻ | 1 | 367.12 | 186.5 | — |
| [α-CD-H]⁻ | 1 | 971.25 | 285.1 | — |
| [β-CD-H]⁻ | 1 | 1133.29 | 314.2 | — |
| [γ-CD-H]⁻ | 1 | 1295.34 | 311.9 | — |
| [α-CD + BPA-H]⁻ | 1 | 1199.41 | 308.1 | 23 |
| [β-CD + BPA-H]⁻ | 1 | 1361.46 | 324.4 | 10.2 |
| [γ-CD + BPA-H]⁻ | 1 | 1523.53 | 349.5 | 37.6 |
| [α-CD + CUR-H]⁻ | 1 | 1339.42 | 324.3 | 39.2 |
| [β-CD + CUR-H]⁻ | 1 | 1501.46 | 330.5 | 16.3 |
| [γ-CD + CUR-H]⁻ | 1 | 1663.51 | 355.7 | 43.8 |

As illustrated in Table 1, it was noticed that the collision cross section of each host-guest complex did not critically increase compared to the collision cross section value of its host molecule. For example, in the case of [α-CD+BPA-H]⁻ complex, the collision cross section was measured to be 308.11 Δ² despite the collision cross section value of [α-CD-H]⁻ 285.1 Å² and [BPA-H]⁻ 160.8 Å². If the complex was not in the host-guest complex form, the estimation collision cross section of the [α-CD+BPA-H]⁻ complex collision cross section could be around 160.8+285.1=345.9 Å². Herein, the collision cross section value of the mentioned complex was measured to be 308.11 Å². The collision cross section value of the complex was found to be only 28 Å² higher than the collision cross section of [α-CD-H]⁻ not the amount of the collision cross section of [BPA-H]⁻ that has 160.8 Å². Similar observations were followed for the other host-guest complexes studied in this work (Table 1). The main observation was that the minimum collision cross section increments of the host-guest complexes compared to the host molecule were found in the case of β-CD. This showed that the guest molecule could be exactly fitted in the suitable cavity of β-CD.

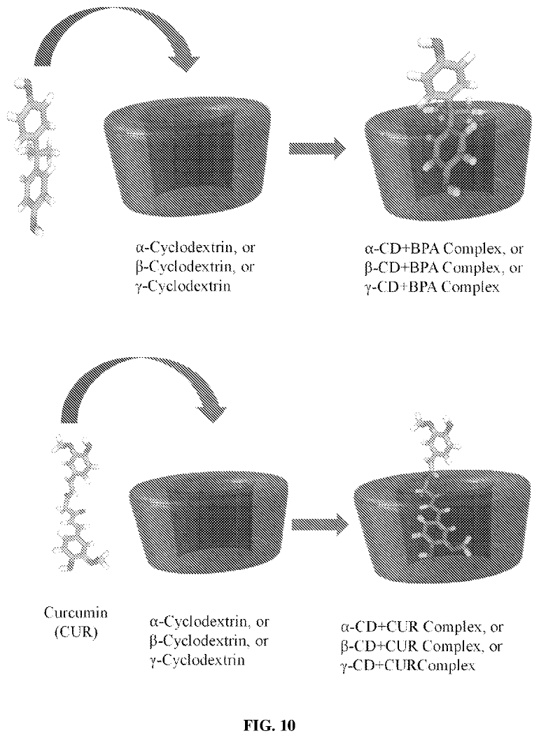

To evaluate the host-guest complex stabilities for the studied complexes, MS/MS analysis was performed at different collision energy (CE) values to obtain the survival yield (SY) of the precursor ions. SY values were calculated for each precursor ion using Equation 1 and Equation 2 given in the experimental part. Schematic representation of BPA- or CUR-CD complexes of CD is given in FIG. 10.

Fifty percent of Center-of-Mass Energy (ECM₅₀ in eV) of the guest-host complexes were calculated and are given in Table 2.

TABLE 2
Overview of the calculated E_{CM50} of the cyclodextrin complexes
and the amount of guest molecules absorbed by CDs plotted
as (mmol/g) and (mmol/mmol) absorbed amount.

| | | Amount of | Amount of |
|---|---|---|---|
| | 50 percent of | Absorbed | Absorbed |
| | Center-of-Mass | Guest | Guest |
| Samples | Energy (E_{CM50}) (eV) | (mmol/g) | (mmol/mmol) |
| [α-CD + BPA-H]⁻ | 0.274 | 0.162 | 0.158 |
| [β-CD + BPA-H]⁻ | 0.272 | 0.245 | 0.279 |
| [γ-CD + BPA-H]⁻ | 0.251 | 0.170 | 0.222 |
| [α-CD + CUR-H]⁻ | 0.505 | 0.494 | 0.482 |
| [β-CD + CUR-H]⁻ | 0.562 | 0.350 | 0.398 |
| [γ-CD + CUR-H]⁻ | 0.508 | 0.320 | 0.416 |

As shown in Table 2, absorbed BPA values were found to be 0.158, 0.279 and 0.222 mmol/mmol for α-, β- and γ-CDs, respectively, and the absorbed CUR values of α-, β- and γ-CDs were found to be 0.482, 0.398 and 0.416 mmol/mmol, respectively. These observations showed that BPA penetrated through the cavity of CDs. On the other hand, CUR rolled up from both sites and penetration of CUR through the cavity of CDs was limited because of its hydrophobic end groups at both sides. For these reasons, CUR penetrated partially into the CDs from both sides resulting in more collision cross section increment, as seen in Table 1, and more absorption capacity per unit CDs.

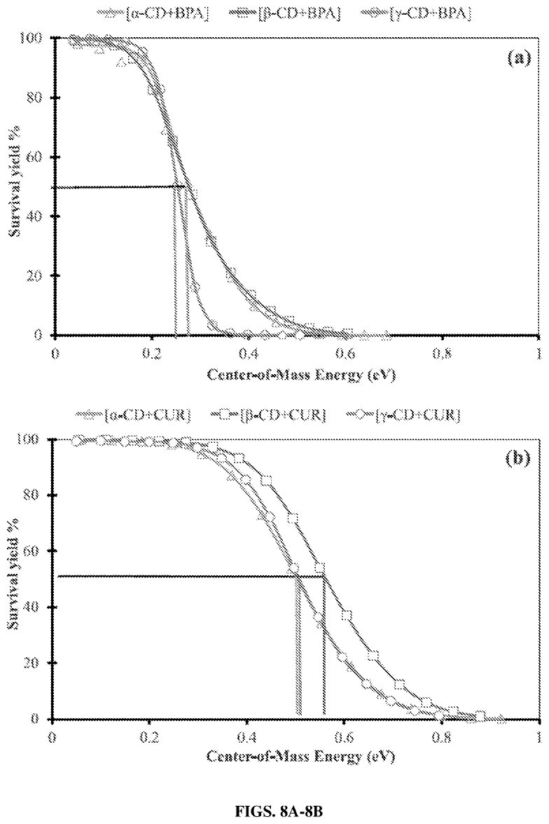

Survival yield curves of BPA or CUR with α-, β- and γ-CDs are given in FIG. 8A-FIG. 8B, respectively.

In the case of BPA, the complexes of α- and β-CD with BPA was found to be more stable compared to γ-CD-BPA complex. On the other hand, in CUR case, the stability of β-CD-CUR complex was found to be higher than the other CD complexes having almost similar complex stability. Therefore, it is presumed that among BPA or CUR complexes with all CDs, the least stable complexes were γ-CD complexes. This is because of the wide internal cavity to weaken the stability of the complexes with BPA and CUR. On the other hand, in every case, the most stable complexes were observed when the β-CD was involved.

Furthermore, to corroborate potential active agent or drug delivery carrier capabilities of p(α-CD), p(β-CD) and p(γ-CD) particles, a carcinogenic compound BPA and a phenolic compound CUR were separately loaded into the p(CD) particles. BPA or CUR loading capacity of p(α-CD), p(β-CD), and p(γ-CD) particles were illustrated in Table 3.

TABLE 3
Bisphenol A (BPA) and curcumin (CUR) loading amounts
p(α-CD) particles, p(β-CD) particles, and p(γ-CD)
particles plotted as mmol/g drug loaded amount.

| | Bisphenol A loading | Curcumin loading |
|---|---|---|
| Materials | (mmol/g) | (mmol/g) |
| p(α-CD) particles | 0.162 | 0.494 |
| p(β-CD) particles | 0.245 | 0.350 |
| p(γ-CD) particles | 0.170 | 0.320 |

As seen from Table 3, the loading capacity of BPA into p(α-CD), p(β-CD), and p(γ-CD) particles were determined as 0.162, 0.245, and 0.170 mmol/g, respectively. Apparently, p(β-CD) particles show the maximum BPA loading amount in comparison to the other CDs particles. Also, CUR as a drug molecule was loaded into p(α-CD), p(β-CD), and p(γ-CD) particle with 0.494, 0.350, and 0.320 mmol/g drug loading amounts, respectively. So, it is obvious that p(α-CD) particles which contain six glucose subunits in the chemical structure afforded the highest amounts of CUR loading. Interestingly, the active agent CUR loading amount was significantly decreased by increasing in the number of glucose subunits to seven and eight for β-CD and γ-CD particles, respectively.

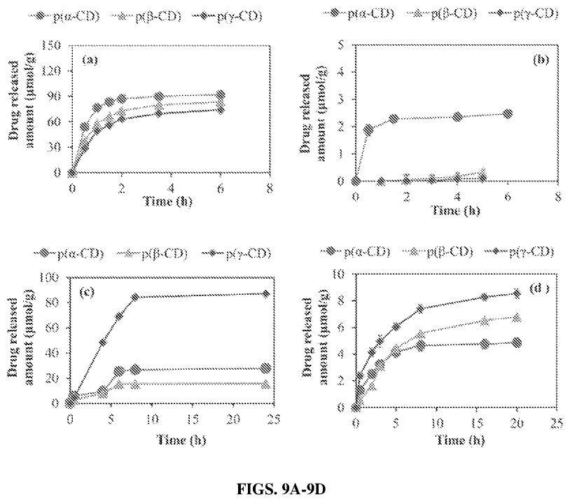

In vitro drug release profiles of BPA and CUR from p(α-CD), p(β-CD), and p(γ-CD) particles are demonstrated in FIG. 9A-FIG. 9D.

In FIG. 9A, the release profile of BPA from p(α-CD), p(β-CD), and p(γ-CD) particles in ethanol solution was demonstrated. P(α-CD), p(β-CD), and p(γ-CD) particles released 91.88±0.1, 83.5±1, and 74.14±2 mmol/g BPA within 6 h in the ethanol solution, which is equal to almost 56%, 34%, and 43% of loaded active agent, respectively. It is obvious that the BPA release from CD-particles is rapid (2 h), and the release rate decreased significantly between 2 to 6 h. On the other hand, in vitro drug release studies of CUR from p(α-CD), p(β-CD), and p(γ-CD) particles showed 2.47±0.1, 0.3±0.05 and 0.09±0.001 mmol/g drug released amount in 6 hours in the ethanol solution, respectively. The release profile of CUR from p(α-CD), p(β-CD), and p(γ-CD) particles was demonstrated in FIG. 9B. It can be seen that the CUR release from p(α-CD) particles in ethanol solution is rapid almost for the first 1 h and the release rate decreased in the 2-6 h range, whereas CUR released amount from p(β-CD), and p(γ-CD) particles were very low, and the release did not have any burst release or flash release characteristics.

In vitro drug release studies of BPA or CUR from p(α-CD), p(β-CD), and p(γ-CD) particles were also performed in PBS at pH 7.4, 37° C. and the corresponding results are presented in FIGS. 9C and 9D, respectively. As seen in FIG. 9C, BPA release from p(α-CD), p(β-CD), and p(γ-CD) particles were determined as 27.69±1, 15.62±0.8, and 87.25±2 mmol/g in 24 h, respectively, whereas p((α-CD), p(β-CD), and p(γ-CD) particles exhibited 4.8±0.5, 6.7±0.3, and 8.55±0.9 mmol/g CUR release in 20 h, respectively. For both BPA and CUR, relatively sustained release profiles were achieved in PBS compared to ethanol solution. The low release profile of CUR can be attributed to its low aqueous solubility at neutral pH. The p(γ-CD) particles, containing 8 glucose units, show the highest BPA and CUR released amount in PBS, compared to other CD particles. As revealed in FIG. 9A and FIG. 9B, p(α-CD) particles show the highest BPA or CUR released amount in ethanol solution. Interestingly, as seen in FIG. 9C and FIG. 9D, p(γ-CD) particles show the highest BPA or CUR released amount in PBS. The different drug release profiles obtained in ethanol and PBS mediums can be attributed to the solubility difference of the loaded agents originating from the internal hydrogen bond networks.

Rapid active agent release from p(α-CD), p(β-CD), and p(γ-CD) particles indicate that these biocompatible drug carrier systems could provide controlled release of therapeutic agents such as CUR or rapid removal of the toxic compounds form contaminated environments such as the much-concerned compound BPA. Additionally, different loading and release profiles of p(α-CD), p(β-CD), and p(γ-CD) particles for BPA and CUR could be attributed to molecular weight and physicochemical properties of these active agents.

Overall, due to their non-toxic nature, good biocompatibility, and high blood compatibility characteristics, p(α-CD), p(β-CD), and p(γ-CD) particles can be functionalized for the delivery of drugs with solubility problems such as curcumin, drugs with high cytotoxic effect e.g., doxorubicin, mitomycin C, mitoxantrone and various other toxic compounds including Bisphenol A, dyes, aromatic phenolics or pesticides. Also, the controlled release of active agents from p(α-CD), p(β-CD), and p(γ-CD) particles prevents the toxic effect of drugs or overdosing of drug-related poisoning of healthy cells in treatments of various diseases such as bacterial and fungal infections, arthritis, neurological diseases, or certain types of cancers that required a continuous and certain level of dosing of drugs for an extended time frame.

### CONCLUSIONS

Herein, p(α-CD), p(β-CD), and p(γ-CD) particles were synthesized in a single step via microemulsion cross-linking method. P(CD) based particles were prepared in one step with only chemical crosslinker using direct cyclodextrin units. The most important advantage of the prepared CD-based particles in comparison to the other kinds of polymeric particles is the use of CD units a monomer that can also accommodate specific molecules such as drugs and active agents in the cavities of the CD units in addition to the particles volumes which is the volume between the CD units. SEM analysis confirmed that prepared cyclodextrin (CD)-based particles are in spherical shape with smooth surfaces in the sizes range of a few hundred nanometers to tens of micrometers. Hemocompatibility analysis of the p(α-CD), p(β-CD) and p(γ-CD) particles was evaluated employing hemolysis and coagulation (blood clotting) assays and estimated that p(α-CD), p(β-CD), p(γ-CD) particles showed immaculate high blood clotting index values at 1 mg/mL concentrations. P(α-CD) and p(β-CD) were found to be non-hemolytic with <2% hemolysis ratios, whereas p(γ-CD) particles were found to be slightly hemolytic materials at 1 mg/mL concentration which is tolerable. The cytotoxicity tests of p(α-CD), p(β-CD) and p(γ-CD) particles on L929 fibroblasts proved that these particles did not cause any significant toxicity up to 1 mg/mL concentrations. Electrospray-Ion Mobility-Mass Spectrometry (ESI-IM-MS) and tandem mass spectrometry (MS/MS) were employed for the complexation of CD with Bisphenol A (BPA) and Curcumin (CUR) and CD-BPA/CUR were analyzed in terms of host-guest complex stabilities. It was concluded that the complexation of α- and β-CD with BPA were more stable compared to the γ-CD-BPA complex. In the case of CUR, the stability of β-CD-CUR complex was found to be higher than the other CD complexes. Therefore, among BPA and CUR complexes with all CDs, the least stable complexes were determined as γ-CD complexes. This is due to the fact that the wide internal cavity of γ-CD can weaken the stability of the complexes with BPA and CUR. As a result, the most stable complexes were observed for β-CD with both BPA and CUR. Moreover, drug loading and release efficiencies of p(α-CD), p(β-CD) and p(γ-CD) particles were evaluated by loading BPA or CUR as active agents and concluded that CD particles depending on the nature (structure) and size of the drug or active agents, they can be used for selective drug loading and promising drug release vehicles with anticipated release profiles. Furthermore, various chemical conjugation methods can be designed to increase the amount of drug loading and/or release that prompts more sustained drug release kinetics. For the dissolution of hydrophobic drugs as well as their bioavailability and stability, the CDs and their corresponding p(α-CD), p(β-CD), and p(γ-CD) particles can deliver afford an outstanding environment for the specific molecules e.g., drugs, toxic organic compounds, certain hormones, lipophilic molecules, proteins, and other biological molecules. The outcomes of these experiments could serve as preliminary inputs for further studies as CDs in particle formulation enhance the bioavailability, efficacy, and stability of therapeutic agents without causing any complications.

### Example 2: Co-polymeric p(cyclodextrin) Particles with Selective and Superior Carrying Abilities of Active Agents

Alpha (α-), beta (β-), and gamma (α-) cyclodextrins (CDs) are macrocyclic oligosaccharides with 6, 7, and 8 glucose moieties, respectively. Although α-, β-, and γ-CDs have a height of torus of 7.8 A, their cavity sizes and volumes increase in the order of α-, β-, and γ-CDs. CDs have rigid truncated cone-like structure with a hydrophilic outer surface and a hydrophobic core. CDs are obtained by the hydrolysis of starch and are biocompatible, biodegradable, and easy to produce. Here, the synthesis and selective active agent loading abilities of poly(Cyclodextrin) and co-polymeric (cyclodextrin) particles are reported.

### Materials

Cyclodextrins, α-cyclodextrin (α-CD, 98%, spectrum chemical MFG CORP), β-cyclodextrin (β-CD, minimum 98%, Sigma), γ-cyclodextrin (γ-CD, <%99, TCI), and the crosslinker divinyl sulfone (DVS, 97%, Merck) were used as received. Ciprofloxacin hydrochloride as an antibiotic (Enzo Biochem, USA), methyl orange (Thermo Scientific, USA) and methylene blue (pure certified, Acros organics, USA) as dyes were used as model active agents.

### Methods

### The Synthesis of Homo Polymeric Cyclodextrin and Co-Polymeric Cyclodextrin Particles

Particles of CD as p(α-Cyclodextrin), p(D-Cyclodextrin) and p(γ-Cyclodextrin) were synthesized via a micro emulsion polymerization method. Briefly, 0.5 g of each α-, β-, or γ-CD monomers were dissolved in 5 mL of 0.25M NaOH in a vial separately as CD solution. Next, 0.5 mL of CD solution was added into a 30 mL of 0.1 M lecithin/cyclohexane medium and stirred at 1000 rpm for 30 min. Following this, 18.2 L of chemical crosslinker, divinyl sulfone (DVS) at 100% mole ratio of total CD was added to the reaction medium and stirred at 1000 rpm for 1

Similarly, co-polymeric (cyclodextrin) particles as p(β-CD-co-γ-CD), pp(α-CD-co-γ-CD), poly(α-CD-co-f-CD) particles were synthesized via micro emulsion polymerization. For this, 0.21 g α-CD and 0.25 g R-CD (1:1 mol ratio), 0.25 g α-CD and 0.33 g γ-CD (1:1 mol ratio) and 0.25 g R-CD and 0.28 g γ-CD (1:1 mol ratio) oligomers were dissolved in 5 mL of 0.25M NaOH in a vial to prepare stock CD solution. After this step, 0.5 mL of stock CD solution was added 30 mL of 0.1 M lecithin/cyclohexane medium and stirred at 1000 rpm for 30 min. Following this, 18.2 μL of chemical crosslinker divinyl sulfone (DVS) at 100% mole ratio of total CD was added to the reaction medium and stirred at 1000 rpm for 1 Then, poly(O-CD-co-γ-CD), poly(α-CD-co-γ-CD), poly(α-CD-co-β-CD) particles were precipitated and washed in 30 mL of ethanol three times, then washed with acetone: DI water 1:1 once, and dried by a heat-gun.

Active agent loading into p(CD) and co-polymeric p(CD) particles Ciprofloxacin as a model antibiotic drug, methylene blue and methyl orange as model active agents were selected for the loading process. For this goal, 20 mL of ciprofloxacin, methylene blue and methyl orange aqueous solutions at 40 ppm concentration were separately prepared in 50 mL falcon tubes. Then, 0.05 g of each p(CD) particles was weighed and placed in the active agent solutions and stirred at 500 rpm 12 h. After this period, active agent-loaded p(CD) particles were precipitated by centrifugation in the same medium at 10,000 rpm for 5 min, then washed once with an ethanol solution to eliminate the active agent molecules that adhered to the outer surfaces. Finally, active agent loaded p(CD) and co-polymeric p(CD) particles were dried at 50° C. oven for 12 h.

In Vitro Drug Release Studies of Ciprofloxacin, Methylene Blue or Methyl Orange Loaded p(CD) and Co-Polymeric p(CD) Particles

In vitro drug release studies from p(CD) and co-polymeric p(CD) particles were done at 37° C. and physiological pH condition, pH 7.4 to mimic the normal body temperature. First, active agent loaded particles weighing 50 mg was suspended in 1 mL of phosphate-buffered saline solution (PBS, sterilized) in dialysis tubing. Then, particle containing dialysis membrane was placed in 40 mL of PBS solution in falcon tubes and kept in a shaking bath. The UV-Vis spectra of 1 mL volume of the samples taken from drug elution media were recorded at various times i.e., 30 min, 1 h, 3 h, 6 h, and each point of measurement were done three times, and the result are given as the average values. The loaded and released amounts of antibiotic drug were calculated by using the calibration curves constructed in PBS for ciprofloxacin, methylene blue and methyl orange at 275, 663 and 464 nm, respectively, via UV-Vis spectroscopy.

### Results

The active agent loading, and release studies of p(CD) and co-polymeric p(CD) particles are given in Table 4 and Table 5, respectively.

TABLE 4
Active agent loading amounts (mg/g) of p(α-CD), p(β-CD) and p(γ-CD)
particles following the 12 h active agent loading period.

| Active agent loading | | | |
|---|---|---|---|
| amount (μg/g) | Ciprofloxacin | Methyl Orange | Methylene Blue |
| P(α-Cyclodextrin) | 512.5 ± 21.8 | 374.7 ± 17.5 | 472.2 ± 48.4 |
| P(β-Cyclodextrin) | 108.8 ± 28.5 | 397.0 ± 28.1 | 808.5 ± 54.3 |
| P(γ-Cyclodextrin) | 1015.9 ± 36.9 | 250.6 ± 10.5 | 448.4 ± 46.9 |

As shown in Table 4, ciprofloxacin loading amounts into p(α-CD), p(β-CD) and p(γ-CD) particles were determined as 512.5±21.8, 108.8±28.5 and 1015.9±36.9 μg/g, respectively. Methyl orange loading amounts of p(α-CD), p(β-CD) and p(γ-CD) particles were determined as 374.7±17.5, 397.0±28.1, 250.6±10.5 μg/g, respectively. Methylene blue loading amounts of p(α-CD), p(β-CD) and p(γ-CD) particles were determined as 472.2±48.4, 808.5±54.3, 448.4±46.9 μg/g, respectively. As seen, ciprofloxacin was found to be highly specific for p(γ-CD) with 2-fold and 10-fold higher drug loading amount in comparison to p(α-CD) and p(β-CD), respectively. Methyl orange was loaded specifically into p(α-CD) and poly(β-CD) particles. Methylene blue showed high specificity for p(β-CD) with 2-fold higher drug loading amount compared to other p(CD) particles.

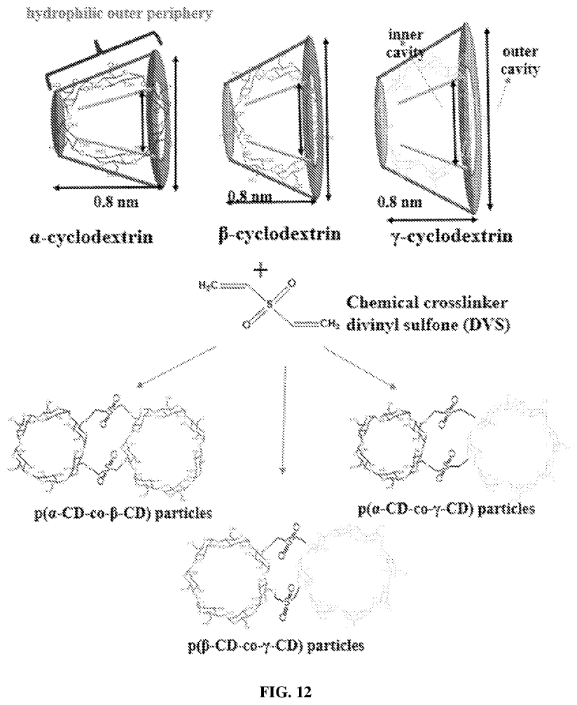

The chemical structure of α-CD, β-CD, and γ-CD and the co-polymeric particles of p(α-CD-co-β-CD), p(α-CD-co-γ-CD) and p(D-CD-co-γ-CD) are given FIG. 12.

TABLE 5
Active agent loading amounts as (μg/g) into p(α-CD-
co-β-CD), p(α-CD-co-γ-CD) and p(β-CD-
co-γ-CD) particles at a 12 h active agent loading period.

| Active agent | | | |
|---|---|---|---|
| loading amount | | | |
| (μg/g) | Ciprofloxacin | Methyl Orange | Methylene Blue |
| P(α-CD-co-β-CD) | 1065.8 ± 44.9 | 1436.7 ± 87.7 | 758.7 ± 19.08 |
| P(α-CD-co-γ-CD) | 2035.8 ± 5.8 | 1146.4 ± 63.2 | 676.7 ± 54.3 |
| P(β-CD-co-γ-CD) | 1689.3 ± 3.2 | 439.2 ± 24.6 | 769.1 ± 39.6 |

As illustrated in Table 5, ciprofloxacin loading amounts into p(α-CD-co-β-CD), p((-CD-co-γ-CD) and p(R-CD-co-γ-CD) particles were determined as 1065.8±44.9, 2035.8±5.8 and 1689.3±3.2 μg/g, respectively. Methyl Orange loading amounts into p(α-CD-co-β-CD), p(α-CD-co-γ-CD) and p(β-CD-co-γ-CD) particles were determined as 1436.7±87.7, 1146.4±63.2, 439.2±24.6 μg/g, respectively. Methylene Blue loading amounts into p(α-CD-co-β-CD), p(α-CD-co-γ-CD) and p(β-CD-co-γ-CD) particles were determined as 758.7±19.08, 676.7±54.3, 769.1±39.6 μg/g, respectively. As seen, ciprofloxacin was adsorbed more into p(α-CD-co-γ-CD) and p(β-CD-co-γ-CD), while it is absorbed in lesser amount in the absence of γ-CD. This result supports that the antibiotic ciprofloxacin can specifically create host-guest complex with γ-CD compared to other CDs. Methyl orange adsorption amounts into p(α-CD-co-β-CD) and p(α-CD-co-γ-CD) were considerably higher than p(β-CD-co-γ-CD), which supports that α-CD can adsorb this dye molecule significantly higher than other CDs. Considering the similar drug loading amount of p(α-CD) and p(β-CD), the highest methyl orange amount of the co-polymeric particle containing these CDs is consistent. Lastly, methylene blue was loaded into p(α-CD-co-β-CD) and p(Q-CD-co-γ-CD) more than p(α-CD-co-γ-CD), which shows that in the absence of α-CD, this dye molecule was less absorbed.

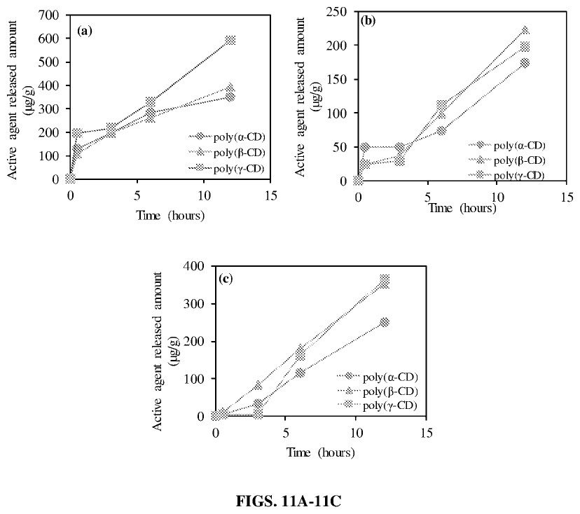

In vitro active agent release from p(α-CD), p(β-CD) and p(γ-CD) particles was performed at 37° C. and pH 7.4 (physiological conditions) and their results are shown in FIG. 11A-FIG. 11C.

As seen in FIG. 11A, ciprofloxacin released amounts of p(α-CD), p(β-CD) and p(γ-CD) particles were determined as 348.8±2.0, 392.4±8.5 and 588.6±6.9 μg/g, respectively. Methyl orange released amounts from p(α-CD), p(β-CD) and p(γ-CD) particles were determined as 173.7±7.5, 223.2±8.1, 198.6±12.5 μg/g, respectively (FIG. 11B). Methylene blue released amounts from p(α-CD), p(β-CD) and p(γ-CD) particles were determined as 249.2±9.4, 351.5±24.3, 364.4±6.9 μg/g, respectively (FIG. 11C). These results show that p(γ-CD) particles released the highest amount of ciprofloxacin within 12 h. Methyl orange released amount of p(β-CD) was the highest and the highest amount of methylene blue release was observed from p(γ-CD) particles. These preliminary results indicate that α-, 0- and γ-CDs with their varied central cavity diameters, 4.7-5.3 A° for α-CD, 6.0-6.5 A° for β-CD and 7.5-8.3 A° for γ-CD, exhibit selective carrying abilities for active agents. The selective adsorption capability of p(α-CD), p(β-CD) and p(γ-CD) particles could be illustrated as a key and lock interaction between host CDs and guest molecules such as drugs, dyes, toxic compounds, active agents, etc. Therefore, p(CD) particles and their co-polymeric forms can used to be loaded many different active ingredients for various purposes to attain higher drug loading efficiency and enhanced adsorption capacity, and release kinetics which are very important especially in the biomedical fields.

Other advantages which are obvious, and which are inherent to the invention, will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

### REFERENCES

K. Kellett, S. A. Kantonen, B. M. Duggan, M. K. Gilson, Toward Expanded Diversity of Host-Guest Interactions via Synthesis and Characterization of Cyclodextrin Derivatives, J. Solution Chem. 47 (2018) 1597-1608. https://doi.org/10.1007/s10953-018-0769-1.
W.-F. Lai, Design of cyclodextrin-based systems for intervention execution, Deliv. Ther. Biogerontological Interv. (2019) 49-59. https://doi.org/10.1016/b978-0-12-816485-3.00005-2.
J. Wankar, N. G. Kotla, S. Gera, S. Rasala, A. Pandit, Y. A. Rochev, Recent Advances in Host-Guest Self-Assembled Cyclodextrin Carriers: Implications for Responsive Drug Delivery and Biomedical Engineering, Adv. Funct. Mater. 30 (2020) 1909049. https://doi.org/10.1002/adfm.201909049.
J. Li, X. J. Loh, Cyclodextrin-based supramolecular architectures: Syntheses, structures, and applications for drug and gene delivery, Adv. Drug Deliv. Rev. 60 (2008) 1000-1017. https://doi.org/10.1016/j.addr.2008.02.011.
S. Demirci, D. Khiev, M. Can, M. Sahiner, M. R. Biswal, R. S. Ayyala, N. Sahiner, Chemically Cross-Linked Poly(β-Cyclodextrin) Particles as Promising Drug Delivery Materials, ACS Appl. Polym. Mater. 3 (2021) 6238-6251. https://doi.org/10.1021/acsapm.1c01058.
T. Loftsson, D. Duchene, Cyclodextrins and their pharmaceutical applications, Int. J. Pharm. 329 (2007) 1-11. https://doi.org/10.1016/j.ijpharm.2006.10.044.
B. G. Poulson, Q. A. Alsulami, A. Sharfalddin, E. F. El Agammy, F. Mouffouk, A.-H. Emwas, L. Jaremko, M. Jaremko, Cyclodextrins: Structural, Chemical, and Physical Properties, and Applications, Polysaccharides. 3 (2021) 1-31. https://doi.org/10.3390/polysaccharides3010001.
A. L. Laza-Knoerr, R. Gref, P. Couvreur, Cyclodextrins for drug delivery, J. Drug Target. 18 (2010) 645-656. https://doi.org/10.3109/10611861003622552.
M. V. Rekharsky, Y. Inoue, Complexation Thermodynamics of Cyclodextrins, Chem. Rev. 98 (1998) 1875-1918. https://doi.org/10.1021/cr970015o.
S. Demirci, D. Khiev, M. Can, M. Sahiner, M. R. Biswal, R. S. Ayyala, N. Sahiner, Chemically Cross-Linked Poly(β-Cyclodextrin) Particles as Promising Drug Delivery Materials, ACS Appl. Polym. Mater. 3 (2021) 6238-6251. https://doi.org/10.1021/acsapm.IcO1058.
F. Topuz, T. Uyar, Advances in the development of cyclodextrin-based nanogels/microgels for biomedical applications: Drug delivery and beyond, Carbohydr. Polym. 297 (2022) 120033. https://doi.org/10.1016/j.carbpol.2022.120033.
F. Bellia, D. La Mendola, C. Pedone, E. Rizzarelli, M. Saviano, G. Vecchio, Selectively functionalized cyclodextrins and their metal complexes, Chem. Soc. Rev. 38 (2009) 2756. https://doi.org/10.1039/b718436k.
A.S.H. Makhlouf, N.Y. Abu-Thabit, eds., Stimuli Responsive Polymeric Nanocarriers for Drug Delivery Applications, Elsevier, 2019. https://doi.org/10.1016/C2016-0-00603-4.
J. D. Buckley, A. M. Coates, P.R.C. Howe, Fiber Ingredients, 1st Editio, 2009.
J. D. Artiss, K. Brogan, M. Brucal, M. Moghaddam, K.-L.C. Jen, The effects of a new soluble dietary fiber on weight gain and selected blood parameters in rats, Metabolism. 55 (2006) 195-202. https://doi.org/10.1016/j.metabol.2005.08.012.
A.K.F.I. Câmara, C. de S. Paglarini, V.A.S. Vidal, M. dos Santos, M.A.R. Pollonio, Meat products as prebiotic food carrier, in: 2020: pp. 223-265. https://doi.org/10.1016/bs.afnr.2020.06.009.
E. Bessell, N. R. Fuller, T. P. Markovic, N. S. Lau, J. Burk, C. Hendy, T. Picone, A. Li, I.D. Caterson, Effects of α-Cyclodextrin on Cholesterol Control and Hydrolyzed Ginseng Extract on Glycemic Control in People With Prediabetes, JAMA Netw. Open. 3 (2020) e2023491. https://doi.org/10.1001/jamanetworkopen.2020.23491.
C. Jen, Grunberger, Artiss, On the binding ratio of &amp;alpha;-cyclodextrin to dietary fat in humans, Nutr. Diet. Suppl. (2013) 9. https://doi.org/10.2147/NDS.S45425.
A. H. Zolnourian, S. Franklin, I. Galea, D. O. Bulters, Study protocol for SFX-01 after subarachnoid haemorrhage (SAS): a multicentre randomised double-blinded, placebo controlled trial, BMJ Open. 10 (2020) e028514. https://doi.org/10.1136/bmjopen-2018-028514.
H. Arima, K. Motoyama, T. Higashi, Cyclodextrin/Dendrimer Conjugates as DNA and Oligonucleotide Carriers, Curr. Top. Med. Chem. 14 (2014) 465-477. https://doi.org/10.2174/1568026613666131219123610.
V. J. Stella, R. A. Rajewski, Sulfobutylether-β-cyclodextrin, Int. J. Pharm. 583 (2020) 119396. https://doi.org/10.1016/j.ijpharm.2020.119396.
K. Hemine, N. Lukasik, M. Gazda, I. Nowak, O-cyclodextrin-containing polymer based on renewable cellulose resources for effective removal of ionic and non-ionic toxic organic pollutants from water, J. Hazard. Mater. 418 (2021) 126286. https://doi.org/10.1016/j.jhazmat.2021.126286.
M.-Y. Xu, H.-L. Jiang, Z.-W. Xie, Z.-T. Li, D. Xu, F.-A. He, Highly efficient selective adsorption of anionic dyes by modified O-cyclodextrin polymers, J. Taiwan Inst.
Chem. Eng. 108 (2020) 114-128. https://doi.org/10.1016/j.jtice.2020.01.005.
S. Swaminathan, R. Cavalli, F. Trotta, P. Ferruti, E. Ranucci, I. Gerges, A. Manfredi, D. Marinotto, P. R. Vavia, In vitro release modulation and conformational stabilization of a model protein using swellable polyamidoamine nanosponges of β-cyclodextrin, J. Incl. Phenom. Macrocycl. Chem. 68 (2010) 183-191. https://doi.org/10.1007/s10847-010-9765-9.
D. H. Schwarz, A. Engelke, G. Wenz, Solubilizing steroidal drugs by β-cyclodextrin derivatives, Int. J. Pharm. 531 (2017) 559-567. https://doi.org/10.1016/j.ijpharm.2017.07.046.
M. Kharat, D. J. McClements, Recent advances in colloidal delivery systems for nutraceuticals: A case study—Delivery by Design of curcumin, J. Colloid Interface Sci. 557 (2019) 506-518. https://doi.org/10.1016/j.jcis.2019.09.045.
E. Morillo, F. Madrid, A. Lara-Moreno, J. Villaverde, Soil bioremediation by cyclodextrins. A review, Int. J. Pharm. 591 (2020) 119943. https://doi.org/10.1016/j.ijpharm.2020.119943.
T. Loftsson, Cyclodextrins in Parenteral Formulations, J. Pharm. Sci. 110 (2021) 654-664. https://doi.org/10.1016/j.xphs.2020.10.026.
P. Saokham, T. Loftsson, γ-Cyclodextrin, Int. J. Pharm. 516 (2017) 278-292. https://doi.org/10.1016/j.ijpharm.2016.10.062.
A. Hamedi, A. Anceschi, A. Patrucco, M. Hasanzadeh, A γ-cyclodextrin-based metal-organic framework (γ-CD-MOF): a review of recent advances for drug delivery application, J. Drug Target. 30 (2022) 381-393. https://doi.org/10.1080/1061186X.2021.2012683.
A. Hamedi, A. Anceschi, F. Trotta, M. Hasanzadeh, F. Caldera, Rapid temperature-assisted synthesis of nanoporous γ-cyclodextrin-based metal-organic framework for selective CO2 adsorption, J. Incl. Phenom. Macrocycl. Chem. 99 (2021) 245-253. https://doi.org/tO.1007/s10847-020-01039-1.
M. P. Abugafy, B. L. Caetano, B. G. Chiari-Andrdo, B. Fonseca-Santos, A. M. do Santos, M. Chorilli, L. A. Chiavacci, Supramolecular cyclodextrin-based metal-organic frameworks as efficient carrier for anti-inflammatory drugs, Eur. J. Pharm. Biopharm. 127 (2018) 112-119. https://doi.org/10.1016/j.ejpb.2018.02.009.
M. Duan, Y. Wang, G. Yang, J. Li, Y. Wan, Y. Deng, Y. Mao, High-level production of γ-cyclodextrin glycosyltransferase in recombinant Escherichia coli BL21 (DE3): culture medium optimization, enzymatic properties characterization, and product specificity analysis, Ann. Microbiol. 70 (2020) 70. https://doi.org/10.1186/s13213-020-01610-8.
L. Wang, S. Chen, J. Wu, Cyclodextrin enhanced the soluble expression of Bacillus clarkii γ-CGTase in Escherichia coli, BMC Biotechnol. 18 (2018) 72. https://doi.org/10.1186/s12896-018-0480-8.
S. Datz, B. Illes, D. G6B1, C. v. Schirnding, H. Engelke, T. Bein, Biocompatible crosslinked β-cyclodextrin nanoparticles as multifunctional carriers for cellular delivery, Nanoscale. 10 (2018) 16284-16292. https://doi.org/10.1039/C8NR02462F.
E. Oliva, D. Mathiron, S. Rigaud, E. Monflier, E. Sevin, H. Bricout, S. Tilloy, F. Gosselet, L. Fenart, V. Bonnet, S. Pilard, F. Djedaini-Pilard, New Lipidyl-Cyclodextrins Obtained by Ring Opening of Methyl Oleate Epoxide Using Ball Milling, Biomolecules. 10 (2020) 339. https://doi.org/10.3390/biom10020339.
M. A. Przybyla, G. Yilmaz, C. R. Becer, Natural cyclodextrins and their derivatives for polymer synthesis, Polym. Chem. 11 (2020) 7582-7602. https://doi.org/10.1039/DOPY01464H.
A. Anceschi, F. Caldera, M. Bertasa, C. Cecone, F. Trotta, P. Bracco, M. Zanetti, M. Malandrino, P. E. Mallon, D. Scalarone, New Poly(β-Cyclodextrin)/Poly(Vinyl Alcohol) Electrospun Sub-Micrometric Fibers and Their Potential Application for Wastewater Treatments, Nanomaterials. 10 (2020) 482. https://doi.org/10.3390/nanol0030482.
A. H. Keshavarz, M. Montazer, N. Soleimani, In situ synthesis of polyamidoamine/D-cyclodextrin/silver nanocomposites on polyester fabric tailoring drug delivery and antimicrobial properties, React. Funct. Polym. 152 (2020) 104602. https://doi.org/10.1016/j.reactfunctpolym.2020.104602.
X. Zhou, X. Li, J. Xu, Y. Cheng, F. Cao, Latanoprost-loaded cyclodextrin microaggregate suspension eye drops for enhanced bioavailability and stability, Eur. J. Pharm. Sci. 160 (2021) 105758. https://doi.org/10.1016/j.ejps.2021.105758.
M. Skiba, F. Bounoure, C. Barbot, P. P. Arnaud, M. Skiba., Development of cyclodextrin microspheres for pulmonary drug delivery., J. Pharm. Pharm. Sci. 8 (2005) 409-418.
S. Bucur, I. Mangalagiu, A. Diacon, A. Mocanu, F. Rizea, R. Somoghi, A. Ghebaur, A. Boscomea, E. Rusen, Novel Chemical Architectures Based on Beta-Cyclodextrin Derivatives Covalently Attached on Polymer Spheres, Polymers (Basel). 13 (2021) 2338. https://doi.org/10.3390/polyml3142338.
E. Mauri, S. M. Giannitelli, M. Trombetta, A. Rainer, Synthesis of Nanogels: Current Trends and Future Outlook, Gels. 7 (2021) 36. https://doi.org/10.3390/gels7020036.
N. Ohshita, K. Motoyama, D. Iohara, F. Hirayama, T. Taharabaru, N. Watabe, Y. Kawabata, R. Onodera, T. Higashi, Polypseudorotaxane-based supramolecular hydrogels consisting of cyclodextrins and Pluronics as stabilizing agents for antibody drugs, Carbohydr. Polym. 256 (2021) 117419. https://doi.org/10.1016/j.carbpol.2020.117419.
R. Anand, M. Malanga, I. Manet, F. Manoli, K. Tuza, A. Aykag, C. Ladaviere, E. Fenyvesi, A. Vargas-Berenguel, R. Gref, S. Monti, Citric acid-γ-cyclodextrin crosslinked oligomers as carriers for doxorubicin delivery, Photochem. Photobiol. Sci. 12 (2013) 1841-1854. https://doi.org/10.1039/c3pp50169 h.
O. Adeoye, I. Bartolo, J. Conceigio, A. B. da Silva, N. Duarte, A. P. Francisco, N. Taveira, H. Cabral-Marques, Pyromellitic dianhydride crosslinked soluble cyclodextrin polymers: Synthesis, lopinavir release from sub-micron sized particles and anti-HIV-1 activity, Int. J. Pharm. 583 (2020) 119356. https://doi.org/10.1016/j.ijpharm.2020.119356.
Y. Q. Huang, C.K.C. Wong, J. S. Zheng, H. Bouwman, R. Barra, B. Wahlstr6 m, L. Neretin, M. H. Wong, Bisphenol A (BPA) in China: A review of sources, environmental levels, and potential human health impacts, Environ. Int. 42 (2012) 91-99. https://doi.org/10.1016/j.envint.2011.04.010.
J. Michalowicz, Bisphenol A—Sources, toxicity and biotransformation, Environ. Toxicol. Pharmacol. 37 (2014) 738-758. https://doi.org/10.1016/j.etap.2014.02.003.
T. T. Schug, A. Janesick, B. Blumberg, J. J. Heindel, Endocrine disrupting chemicals and disease susceptibility, J. Steroid Biochem. Mol. Biol. 127 (2011) 204-215. https://doi.org/10.1016/j.jsbmb.2011.08.007.
J. D. Meeker, A. M. Calafat, R. Hauser, Urinary Bisphenol A Concentrations in Relation to Serum Thyroid and Reproductive Hormone Levels in Men from an Infertility Clinic, Environ. Sci. Technol. 44 (2010) 1458-1463. https://doi.org/10.1021/es9028292.
J. R. Rochester, Bisphenol A and human health: A review of the literature, Reprod. Toxicol. 42 (2013) 132-155. https://doi.org/10.1016/j.reprotox.2013.08.008.
G. Schönfelder, W. Wittfoht, H. Hopp, C. E. Talsness, M. Paul, 1. Chahoud, Parent bisphenol A accumulation in the human maternal-fetal-placental unit., Environ. Health Perspect. 110 (2002). https://doi.org/10.1289/ehp.110-1241091.
R. Ahsan, M. Arshad, M. Khushtar, M. A. Ahmad, M. Muazzam, M. S. Akhter, G. Gupta, M. Muzahid, A Comprehensive Review on Physiological Effects of Curcumin, Drug Res. (Stuttg). 70 (2020) 441-447. https://doi.org/10.1055/a-1207-9469.
B. Zheng, D. J. McClements, Formulation of More Efficacious Curcumin Delivery Systems Using Colloid Science: Enhanced Solubility, Stability, and Bioavailability, Molecules. 25 (2020) 2791. https://doi.org/10.3390/molecules25122791.
S. S. Suner, M. Sahiner, A. S. Yilmaz, R. S. Ayyala, N. Sahiner, Light-Activated Modified Arginine Carbon Dots as Antibacterial Particles, Catalysts. 12 (2022) 1376. https://doi.org/10.3390/catal12111376.
T. Mosmann, Rapid colorimetric assay for cellular growth and survival: Application to proliferation and cytotoxicity assays, J. Immunol. Methods. 65 (1983) 55-63. https://doi.org/10.1016/0022-1759(83) 90303-4.
P. Chakraborty, A. Baksi, E. Khatun, A. Nag, A. Ghosh, T. Pradeep, Dissociation of Gas Phase Ions of Atomically Precise Silver Clusters Reflects Their Solution Phase Stability, J. Phys. Chem. C. 121 (2017) 10971-10981. https://doi.org/10.1021/acs.jpcc.6b12485.
J. C. Amicangelo, P. B. Armentrout, Absolute Binding Energies of Alkali-Metal Cation Complexes with Benzene Determined by Threshold Collision-Induced Dissociation Experiments and ab Initio Theory, J. Phys. Chem. A. 104 (2000) 11420-11432. https://doi.org/10.1021/jp002652f.
I. Gomez, Y. Alesanco, J. A. Blizquez, A. Vinuales, L. C. Colmenares, Room-Temperature Self-Standing Cellulose-Based Hydrogel Electrolytes for Electrochemical Devices, Polymers (Basel). 12 (2020) 2686. https://doi.org/10.3390/polymt21t2686.
I. Shown, S. Banerjee, A. V. Ramchandran, K. E. Geckeler, C. N. Murthy, Synthesis of Cyclodextrin and Sugar-Based Oligomers for the Efavirenz Drug Delivery, Macromol. Symp. 287 (2010) 51-59. https://doi.org/10.1002/masy.201050108.
A. Celebioglu, T. Uyar, Electrospinning of Polymer-free Nanofibers from Cyclodextrin Inclusion Complexes, Langmuir. 27 (2011) 6218-6226. https://doi.org/t0.1021/1a1050223.
W. van Oeveren, Obstacles in Haemocompatibility Testing, Scientifica (Cairo). 2013 (2013) 1-14. https://doi.org/10.1155/2013/392584.
B. T. Kurien, A. Singh, H. Matsumoto, R. H. Scofield, Improving the Solubility and Pharmacological Efficacy of Curcumin by Heat Treatment, Assay Drug Dev. Technol. 5 (2007) 567-576. https://doi.org/10.1089/adt.2007.064.
C. Carvalho, R. Santos, S. Cardoso, S. Correia, P. Oliveira, M. Santos, P. Moreira, Doxorubicin: The Good, the Bad and the Ugly Effect, Curr. Med. Chem. 16 (2009) 3267-3285. https://doi.org/10.2174/092986709788803312.
D. R. Hardten, T. W. Samuelson, Ocular Toxicity of Mitomycin-C, Int. Ophthalmol. Clin. 39 (1999) 79-90. https://doi.org/10.1097/00004397-199903920-00007.
LiverTox: Clinical and Research Information on Drug-Induced Liver Injury, Bethesda(MD): National Institute of Diabetes and Digestive and Kidney Diseases, 2012.
Suner, S. S.; Sahiner, M.; Yilmaz, A. S.; Ayyala, R. S.; Sahiner, N. Light-Activated Modified Arginine Carbon Dots as Antibacterial Particles. Catalysts 2022, 12, 1376, doi:10.3390/catal12111376.

1. A polymer comprising repeating units of a cyclodextrin comprising γ-cyclodextrin (γ-CD), or any combination thereof, and a crosslinker.

2. The polymer of claim 1, wherein the cyclodextrin further comprises α-cyclodextrin (α-CD) and/or β-cyclodextrin (β-CD).

3. The polymer of claim 1, wherein the polymer is a reaction product of the cyclodextrin and the crosslinker, wherein the crosslinker comprises an epoxide, a polyacrylic ester, a polycarbonate acid, a polyamidoamine, a tetrafluoroterephthalatenitrile (TFTN), divinyl sulfone (DVS), trimethylolpropanetriglycidyl ether (TMPGDE), or any combination thereof.

4. The polymer of claim 3, wherein the crosslinker comprises DVS or TMPGDE.

5.-9. (canceled)

10. The polymer of claim 2, wherein the cyclodextrin comprises α-CD and γ-CD and the crosslinker comprises DVS or TMPGDE.

11. (canceled)

12. The polymer of claim 2, wherein the cyclodextrin comprises β-CD and γ-CD and the crosslinker comprises DVS or TMPGDE.

13-15. (canceled)

16. The polymer of claim 2, wherein the cyclodextrin comprises α-CD, β-CD, and γ-CD and the crosslinker comprises DVS or TMPGDE.

17. (canceled)

18. A particle comprising the polymer of claim 1.

19. The particle of claim 18, wherein the particle further comprises a therapeutically effective amount of a drug.

20. The particle of claim 19, wherein the drug is hydrophobic.

21. The particle of claim 20, wherein the hydrophobic drug comprises ciprofloxacin, nitrofurantoin, propranolol, a camphotosin analog, silbylin, docetaxel, doxorubicin, naproxen, trimethoprim/sulfamethoxazole, or any combination thereof.

22. The particle of claim 18, wherein the particle further comprises an active agent, wherein the active agent comprises a dye, a toxic compound, a phenolic compound, or any combination thereof.

23. (canceled)

24. The particle of claim 22, wherein:
the phenolic compound comprises curcumin, bisphenol A, chlorophenol, aminophenol, chlorocatechol, nitrophenol, methylphenol, or any combination thereof:
the toxic compound comprises fluorinated chemicals, phthalates, polychlorinated biphenyls, or any combination thereof; and/or
the dye comprises methyl orange, methylene blue, methyl red, Evans blue, gentian violet, crystal violet, safranin, Eosin Y, fuchsine, acid fuchsin, carmine, acridine orange, or any combination thereof.

25-27. (canceled)

28. A cryogel comprising the polymer of claim 1.

29. The cryogel of claim 28, wherein the cryogel further comprises a therapeutically effective amount of a drug.

30. The cryogel of claim 29, wherein the drug is hydrophobic.

31. The cryogel of claim 30, wherein the hydrophobic drug comprises ciprofloxacin, nitrofurantoin, propranolol, a camphotosin analog, silbylin, docetaxel, doxorubicin, naproxen, trimethoprim/sulfamethoxazole, or any combination thereof.

32. The cryogel of claim 28, wherein the cryogel further comprises an active agent, wherein the active agent comprises a dye, a toxic compound, a phenolic compound, or any combination thereof.

33. (canceled)

34. The cryogel of claim 32, wherein:
the phenolic compound comprises curcumin, bisphenol A, chlorophenol, aminophenol, chlorocatechol, nitrophenol, methylphenol, or any combination thereof;
the toxic compound comprises fluorinated chemicals, phthalates, polychlorinated biphenyls, or any combination thereof; and/or
the dye comprises methyl orange, methylene blue, methyl red, Evans blue, gentian violet, crystal violet safranin, Eosin Y, fuchsine acid fuchsin carmine acridine orange or any combination thereof.

35.-39. (canceled)
